(19) United States
(12) Patent Application Publication
(10) Pub. No.: US 2016/0081592 A1
(43) Pub. Date: Mar. 24, 2016

(54) BED HAVING LOAD DETECTION FUNCTION AND BED-LOAD DETECTOR

(71) Applicant: SHOWA DENKO K.K., Tokyo (JP)

(72) Inventors: Motoki ISHIKAWA, Oyama-shi, Tochigi (JP); Kazuo HIROSE, Oyama-shi (JP); Shingo NOGUCHI, Oyama-shi (JP)

(73) Assignee: SHOWA DENKO K.K., Tokyo (JP)

(21) Appl. No.: 14/786,801

(22) PCT Filed: Apr. 24, 2014
(86) PCT No.: PCT/JP2014/061549

§ 371 Date: Oct. 23, 2015

(30) Foreign Application Priority Data
Apr. 26, 2013 (JP) 2013-094606

(51) Int. Cl. A61B 5/11; A61G 7/05

(52) U.S. Cl. CPC A61B 5/1116; A61G 7/05; A61G 2007/0527

(57) ABSTRACT

In a bed having a load detection function, a load detector attached to a bed main body includes a load cell provided at a site which is positioned at any location on a load transmission path extending from a bed surface-forming portion to a bed installation surface, and at which a load from the bed surface-forming portion side is received and the load is transmitted to a configuration member of the installation surface side. The load cell includes a load-receiving member, and a base body which is separated from the load-receiving member and to which the load from the load-receiving member is applied. The load-receiving member comes into contact with the base body, the load from the load-receiving member acts on the base body, and the resulting strain is detected by a strain sensor.

### BACKGROUND OF THE INVENTION

1. Field of the Invention

The present invention relates to a bed having a load detection function which detects a change of a load applied to a bed main body by a load detector attached to the bed main body and detects a state of a user on a bed surface of the bed main body, and a load detector for applying a load detection function to a bed.

Priority is claimed on Japanese Patent Application No. 2013-094606, filed Apr. 26, 2013, the content of which is incorporated herein by reference.

2. Description of Related Art

For example, in a bed which is used in a medical institution, nursing facilities, care facilities, lodging facilities, an ordinary household, or the like, a method has been suggested which detects a change of a load applied to a bed main body, and detects a state (having got into a bed, having got out of a bed, an in-bed position, a movement of a body, or the like) of a user (a sick person, a person who needs care, infants, a healthy person, or the like) on a bed surface of a bed main body (for example, refer to PTLs 1 to 3).

Specifically, PTL 1 discloses a method which disposes a load sensor between a leg portion provided on a bed main body and an installation surface (a floor surface or the like) on which the bed main body is installed, and detects a bed occupancy state of a person based on electric signals from the load sensor. In addition, a slope portion for introducing a caster provided on the leg portion of the bed main body from the installation surface of the bed main body onto a load-receiving portion of the load sensor is formed on the load sensor.

Meanwhile, PTL 2 discloses a method which provides a load detector in a space between a bed main body and an installation surface on which the bed main body is installed and detects a load applied to the bed main body. In addition, in the load detector, means for lifting a bed is provided.

### PRIOR ART DOCUMENTS

### Patent Documents

[PTL 1] Japanese Unexamined Patent Application, First Publication No. 2000-105884

[PTL 2] Japanese Unexamined Patent Application, First Publication No. 2008-304397

[PTL 3] Japanese Unexamined Patent Application, First Publication No. 2007-256074

### SUMMARY OF THE INVENTION

However, in the invention disclosed in PTL 1, when a load of the bed main body is detected using the load sensor, after the caster provided in the leg portion of the bed moves to the vicinity of a front side of the slope portion of the load sensor and passes through the slope portion, the caster should be placed on the load-receiving portion of the load sensor, which is significantly troublesome.

Meanwhile, in the invention disclosed in PTL 2, for example, when the bed main body is installed along a wall, since a provider cannot enter a portion between the bed main body and the wall, it is significantly difficult to dispose the load detector in a space between the bed main body and the installation surface.

In addition, in the invention disclosed in PTL 3, even when the load detector is incorporated into the bed main body in advance, the bed main body should be designed in accordance with the load detector. Accordingly, new parts are required. Therefore, the bed having a load detection function becomes significantly expensive. In addition, the number of parts increases and it is difficult to achieve reduction of weight.

The present invention is made in consideration of the circumstances of the related art, and object thereof is to provide a bed having a load detection function in which a load detection function is capable of being added using a simple structure while preventing an increase in the number of parts, and a bed-load detector capable of being simply and easily incorporated into a bed main body in order to add the load detection function to an existing bed. In addition, another object of the present invention is to provide a bed having a load detection function and a bed-load detector capable of detecting a load with high accuracy without decreasing detection accuracy of a load in a vertically downward direction which is detected initially even when a load applied to a bed surface is greatly biased (an unbalanced load state is generated) due to a user or the like on a bed sitting on an end portion of a bed surface of the bed, a user who lies on a bed surface mainly having turned over toward one end portion side on the bed surface, or the like, and the bed is distorted.

In order to achieve above-described objects, the present invention provides aspects described in the following (1) to (19).

(1) A bed having a load detection function which detects a change of a load applied to a bed main body by a load detector attached to the bed main body and detects a state of a user on a bed surface of the bed main body,

in which the bed main body is configured to include a bed surface-forming portion which forms the bed surface, a leg portion which comes into contact with an installation surface on which the bed main body is to be installed, and a connection-support portion which connects the bed surface-forming portion and the leg portion such that the bed surface-forming portion is positioned above the installation surface, and transmits a load from the bed surface-forming portion to the leg portion,

in which the load detector includes a load cell which measures strain generated when a load is applied to the bed main body,

in which the load cell is provided at a site which is positioned at any location on a load transmission path extending from the bed surface-forming portion to the installation surface via the connection-support portion, and at which a load from the bed surface-forming portion side is received and the load is transmitted to a configuration member of the installation surface side,

in which the load cell includes a load-receiving member which receives a load from the bed surface-forming portion side, and a base body which is separated from the load-receiving member and to which the load from the load-receiving member is applied, and

in which the base body is configured to include an action portion with which the load-receiving member comes into contact and on which a load from the load-receiving member acts, an operating portion which is strain-deformed by the load applied to the action portion, a strain sensor which is attached to the operating portion, and an attachment portion which is continuous with the operating portion and is fixed to the configuration member of the installation surface side in the bed main body.

In the aspect of (1), the term “load transmission path” corresponds to a structural member which supports a load applied to the bed surface-forming portion between the bed surface-forming portion and the installation surface, and may be a structural member through which a load applied to the bed surface-forming portion is transmitted to the leg portion which comes into contact with the installation surface. Accordingly, for example, the “load transmission path” may be configured of the connection-support portion and the leg portion, or the connection-support portion, the leg portion, the bed surface-forming portion, and the caster.

In addition, in the aspect of (1), as a specific aspect of “the load cell being provided at a site which is positioned at any location on the load transmission path and at which a load from the bed surface-forming portion side is received and the load is transmitted to the installation surface side”, preferably, in a state where an arbitrary surface (division surface) by which the load transmission path of the bed main body is vertically divided into the bed surface-forming portion side and the installation surface side is assumed, the load cell is provided on at least one location on a structural member penetrating the division surface vertically. For example, a surface on which a spindle and a bearing portion receiving the spindle come into contact with each other in the load transmission path of the bed main body may be assumed as the division surface, and in this case, the spindle and the bearing portion correspond to structural members penetrating the division surface vertically.

(2) In the bed having a load detection function of the aspect of (1),

the operating portion in the base body of the load cell is configured of a flexibly deformable cantilever portion in which one end is continuous with the action portion and the other end is continuous with the attachment portion.

(3) In the bed having a load detection function of the aspect of (1),

the operating portion in the base body of the load cell is a compression-deformable member in which one end is continuous with the action portion and the other end is continuous with the attachment portion.

(4) In the bed having a load detection function of the aspect of (1),

a spindle having a substantially horizontal axial line is placed in the load transmission path of the bed main body, and a bearing portion which rotatably supports the spindle is formed on the load-receiving member of the base body.

In the aspect of (4), the spindle having a substantially horizontal axial line provided in the load transmission path of the bed main body may be slightly inclined from a horizontal direction. Specifically, for example, due to error when the bed main body is manufactured, inclination or unevenness of the installation surface, a change over time according to use for a long period of time of a bed, a movement of a bed user on a bed surface, or the like, strictly, the spindle may be slightly inclined from a horizontal direction, for example, may be inclined within approximately 5°.

(5) In the bed having a load detection function of the aspect of (1),

the connection-support portion of the bed main body includes a lower frame which is substantially parallel with the installation surface, the leg portion is provided on the lower frame, the lower frame is a configuration member of the installation surface side, and the cylindrical support body is fixed to the lower frame.

(6) In the bed having a load detection function of the aspect of (1),

a hollow cylindrical support body is perpendicularly fixed to the configuration member of the installation surface side in the bed main body such that one end opening portion of the hollow cylindrical support body faces upward, a lower portion of the load-receiving member is inserted into an opening portion of the cylindrical support body from above, and at least a portion of the base body is inserted into a lower portion of the cylindrical support body such that the action portion of the base body is positioned inside the cylindrical support body and the action portion and the operating portion do not come into contact with the cylindrical support body.

(7) In the bed having a load detection function of the aspect of (2),

a hollow cylindrical support body is perpendicularly fixed to the configuration member of the installation surface side in the bed main body such that one end opening portion of the hollow cylindrical support body faces upward, a lower portion of the load-receiving member is inserted into an opening portion of the cylindrical support body from above, an opening window portion is formed on a side surface side of a lower portion of the cylindrical support body, a portion of the base body is inserted into the cylindrical support body from the opening window portion such that at least the action portion is positioned inside the cylindrical support body and the action portion and the operating portion do not come into contact with the cylindrical support body, and the remaining portion of the base body is positioned outside the cylindrical support body.

(8) In the bed having a load detection function of the aspect of (7),

at least a portion of the attachment of the base body is inserted into the cylindrical support body, and the attachment portion is fixed to an inner wall surface of the cylindrical support body.

(9) In the bed having a load detection function of the aspect of (7),

at least a portion of the attachment of the base body is inserted into the cylindrical support body, and the attachment portion is fixed to an inner wall surface of the cylindrical support body.

(10) In the bed having a load detection function of any one of the aspects of (7) to (9),

the connection-support portion of the bed main body includes a lower frame which is substantially parallel with the installation surface, the leg portion is provided on the lower frame, the lower frame is configured by combining at least four pipes, one pipe or two or more pipes are configuration members of the installation surface side, the cylindrical support body is fixed to one pipe or each of two or more pipes, and the base body is disposed such that a length direction of the cantilever portion is along a length direction of a pipe.

(11) In the bed having a load detection function of any one of the aspects of (1) to (4),

the load cell is placed at an intermediate portion of the connection-support portion.

(12) In the bed having a load detection function of the aspect of (11),

the connection-support portion includes a lifting-lowering link mechanism which lifts and lowers the bed surface-forming portion, and

the load cell is incorporated into the lifting-lowering link mechanism.

(13) In the bed having a load detection function of the aspect of (11),

the connection-support portion includes a lower frame which is supported above the installation surface via the leg portion, in addition to the lifting-lowering mechanism, and

the lifting-lowering link mechanism includes at least a first connection arm and a second connection arm as an arm which connects the bed surface-forming portion and the lower frame, the second arm is connected to the bed surface-forming portion side, the first arm is connected to the lower frame side, and the load cell is interposed between the bed surface-forming portion and the lower frame.

(14) In the bed having a load detection function of any one of the aspects of (1) to (3),

the load cell is interposed between the bed surface-forming portion and the connection-support portion.

(15) In the bed having a load detection function of any one of the aspects of (1) to (3),

the load cell is interposed between the connection support portion and the leg portion.

(16) In the bed having a load detection function of any one of the aspects of (1) to (3),

the load cell is incorporated into the leg portion.

(17) In the bed having a load detection function of any one of the aspects of (1) to (3),

the leg portion includes a caster mechanism, and the load cell is incorporated into the caster mechanism.

(18) A bed-load detector which measures a change of a load applied to a bed main body by the bed-load detector being attached to the bed main body and detects a state of a user on a bed surface of the bed main body,

in which the bed main body includes

a bed surface-forming portion which forms the bed surface,

a leg portion which comes into contact with an installation surface on which the bed main body is to be installed, and

a connection-support portion which connects the bed surface-forming portion and the leg portion such that the bed surface-forming portion is positioned above the installation surface, and transmits a load from the bed surface-forming portion to the leg portion,

in which the load detector includes a load cell which measures strain generated when a load is applied to the bed main body,

in which the load cell is configured to be placed at a site which is positioned at any location on a load transmission path extending from the bed surface-forming portion to the installation surface via the connection-support portion, and at which a load from the bed surface-forming portion side is received and the load is transmitted to a configuration member of the installation surface side,

in which the load cell includes a load-receiving member which receives a load from the bed surface-forming portion side, and a base body which is separated from the load-receiving member and to which the load from the load-receiving member is applied, and

in which the base body is configured to include an action portion with which the load-receiving member comes into contact and on which a load from the load-receiving member acts, an operating portion which is strain-deformed by the load applied to the action portion, a strain sensor which is attached to the operating portion, and an attachment portion which is continuous with the operating portion and is fixed to the configuration member of the installation surface side in the bed main body.

Similarly to the above-described aspect of (1), in the aspect of (18), the “load transmission path” corresponds to a structural member which supports a load applied to the bed surface-forming portion between the bed surface-forming portion and the installation surface, and, for example, corresponds to a structural member through which a load applied to the bed surface-forming portion is transmitted to the leg portion which comes into contact with the installation surface, the leg portion itself, a caster which is attached to the leg portion, or the like.

In addition, in the aspect of (18), a specific aspect of “the load cell being placed at a site which is positioned at any location on a load transmission path extending from the bed surface-forming portion to the installation surface via the connection-support portion, and at which a load from the bed surface-forming portion side is received and the load is transmitted to a configuration member of the installation surface side” is similar to that of the aspect of (1).

(19) In the bed-load detector of the aspect of (18),

a bearing portion which rotatably supports a spindle, which is provided in the load transmission path of the bed main body and has a substantially horizontal axial line, is formed on the load-receiving member of the base body.

Moreover, in the aspect of (19), “a spindle having a substantially horizontal axial line” is similar to that of the above-described aspect of (4).

According to the bed having a load detection function of the present invention, it is possible to provide a bed having a load detection function in which a load detection function is capable of being added using a simple structure while preventing an increase in the number of parts and of detecting a load with high accuracy while securing durability of a load detector, and a load detector capable of being separately incorporated into a bed main body in order to add the load detection function to an existing bed. In addition, in the present invention, it is possible to provide a bed having a load detection function and a bed-load detector capable of detecting a load with high accuracy without decreasing detection accuracy of a load in a vertically downward direction to be detected originally even when a load applied to a bed surface is greatly biased (an unbalanced load state is generated) due to a user or the like on a bed sitting on an end portion of a bed surface of the bed, a user who lies on a bed surface largely turning over toward one end portion side on the bed surface, or the like, and the bed is distorted.

### BRIEF DESCRIPTION OF THE DRAWINGS

FIG. 1 is a side view showing an example of a bed having a load detection function to which the present invention is applied.

FIG. 2A is a side view of a main portion of a bed main body showing a state where a bed plate had been lowered by a lifting-lowering link mechanism in a bed shown in FIG. 1.

FIG. 2B is a side view of a main portion of a bed main body showing a state where a bed plate has been lifted by a lifting-lowering link mechanism in a bed shown in FIG. 1.

FIG. 3A is an enlarged side view of a main portion of the lifting-lowering link mechanism into which a load cell is incorporated in the bed shown in FIG. 1.

FIG. 3B is an enlarged front view of a main portion of the lifting-lowering link mechanism into which the load cell is incorporated in the bed shown in FIG. 1.

FIG. 4 is a perspective view showing a lower frame to which the load cell is attached in the bed shown in FIG. 1.

FIG. 5 is an enlarged perspective view of a main portion (a portion indicated by a reference numeral V of FIG. 4) of FIG. 4.

FIG. 6A is a perspective view showing a first example of a load-receiving member of the load cell used in the bed having a load detection function of the present invention.

FIG. 6B is a side view of the load-receiving member shown in FIG. 6A.

FIG. 6C is a sectional view taken along line VIC-VIC of FIG. 6B.

FIG. 7A is a perspective view showing a first example of a base body of the load cell used in the bed having a load detection function of the present invention.

FIG. 7B is a plan view of the base body shown in FIG. 7A.

FIG. 7C is a sectional view taken along line VIIC-VIIC of FIG. 7B.

FIG. 8 is a cross-sectional side view showing a state in which a load cell, which is obtained by combining the load-receiving member of the first example shown in FIGS. 6A to 6C and the base body of the first example shown in FIGS. 7A to 7C, is attached to the bed main body.

FIG. 9A is a side view of an example of an operating portion (cantilever portion) of the base body in the load cell.

FIG. 9B is a top view of an example of the operating portion (cantilever portion) of the base body in the load cell.

FIG. 9C is a circuit diagram showing a Wheatstone bridge circuit of a strain gauge used in the load cell.

FIG. 10 is a view showing a change of the load cell before and after a load is applied to the bed main body and is a cross-sectional side view corresponding to FIG. 8.

FIG. 11A is a perspective view showing a second example of the base body of the load cell used in the bed having a load detection function of the present invention.

FIG. 11B is a plan view of the base body of the second example shown in FIG. 11A.

FIG. 11C is a sectional view taken along line XIC-XIC of FIG. 11B.

FIG. 11D is a bottom view of the base body of the load cell shown in FIG. 11A.

FIG. 12 is a side view showing a state in which a load cell, which is obtained by combining the load-receiving member of the first example shown in FIGS. 6A to 6C and the base body of the second example shown in FIGS. 11A to 11C, is attached to the bed main body.

FIG. 13 is a cross-sectional side view with respect to FIG. 12.

FIG. 14A is a side view showing a second example of the load-receiving member of the load cell used in the bed having a load detection function of the present invention.

FIG. 14B is a sectional view taken along line XIVB-XIVB of FIG. 14A.

FIG. 14C is a perspective view of the second example of the load-receiving member of the load cell shown in FIG. 14A.

FIG. 15A is a cross-sectional side view showing a state in which a load cell, which is obtained by combining the load-receiving member of the second example shown in FIGS. 14A to 14C and the base body of the second example shown in FIGS. 11A to 11C, is attached to the bed main body.

FIG. 15B is a sectional view taken along line XVB-XVB of FIG. 15A.

FIG. 16A is a side view showing a third example of the load-receiving member of the load cell used in the bed having a load detection function of the present invention.

FIG. 16B is a sectional view taken along line XVIB-XVIB of FIG. 16A.

FIG. 16C is a sectional view taken along line XVIC-XVIC of FIG. 16B.

FIG. 17A is a cross-sectional side view showing a state in which a load cell, which is obtained by combining the load-receiving member of the third example shown in FIGS. 16A to 16C and the base body of the second example shown in FIGS. 11A to 11C, is attached to a bed.

FIG. 17B is a sectional view taken along line XVIIB-XVIIB of FIG. 16A.

FIG. 18A is a side view showing a fourth example of the load-receiving member of the load cell used in the bed having a load detection function of the present invention.

FIG. 18B is a sectional view taken along line XVIIIB-XVIIIB of FIG. 18A.

FIG. 18C is a perspective view when viewed from a bottom surface side of the load-receiving member shown in FIG. 18A.

FIG. 19A is a cross-sectional side view showing a state in which a load cell, which is obtained by combining the load-receiving member of the fourth example shown in FIGS. 18A to 18C and the base body of the second example shown in FIGS. 11A to 11C, is attached to the bed main body.

FIG. 19B is a sectional view taken along line XIXB-XIXB of FIG. 19A.

FIG. 20A is a side view showing a fifth example of the load-receiving member of the load cell used in the bed having a load detection function of the present invention.

FIG. 20B is a sectional view taken along line XXB-XXB of FIG. 20A.

FIG. 21A is a side view showing a state in which a load cell, which is obtained by combining the load-receiving member of the fifth example shown in FIGS. 20A and 20B and the base body of the second example shown in FIGS. 11A to 11C, is attached to the bed main body.

FIG. 21B is a sectional view taken along line XXIB-XXIB of FIG. 21A.

FIG. 22 is a perspective view showing a sixth example of the load-receiving member of the load cell used in the bed having a load detection function of the present invention.

FIG. 23A is a front view showing a state in which a load cell, which is obtained by combining the load-receiving member of the sixth example shown in FIG. 22 and the base body of the second example shown in FIGS. 11A to 11C, is attached to the bed main body.

FIG. 23B is a right-side view with respect to FIG. 23A.

FIG. 23C is a sectional view taken along line XXIIIC-XXIIIC of FIG. 23B.

FIG. 23D is a sectional view taken along line XXIIID-XXIIID of FIG. 23C.

FIG. 24A is a side view showing a seventh example of the load-receiving member of the load cell used in the bed having a load detection function of the present invention.

FIG. 24B is a sectional view taken along line XXIVB-XXIVB of FIG. 24A.

FIG. 24C is a sectional view taken along line XXIVC-XXIVC of FIG. 24B.

FIG. 25A is a cross-sectional side view showing a state in which a load cell, which is obtained by combining the load-receiving member of the seventh example shown in FIGS. 24A to 24C and the base body of the second example shown in FIGS. 11A to 11C, is attached to the bed main body.

FIG. 25B is a sectional view taken along line XXVB-XXVB of FIG. 24A.

FIG. 26 is a perspective view showing a third example of the base body of the load cell used in the bed having a load detection function of the present invention.

FIG. 27 is a cross-sectional side view showing a state in which a load cell, which is obtained by combining the load-receiving member of the first example shown in FIGS. 6A to 6C and the base body of the third example shown in FIG. 26, is attached to the bed main body.

FIG. 28 is a side view showing another example of the bed having a load detection function to which the load detector of the present invention is applied.

FIG. 29 is a side view showing still another example of the bed having a load detection function to which the load detector of the present invention is applied.

FIG. 30 is a side view showing still another example of the bed having a load detection function to which the load detector of the present invention is applied.

### DETAILED DESCRIPTION OF THE INVENTION

Hereinafter, embodiments of a bed having a load detection function and a load detector to which the present invention is applied will be described with reference to the drawings. In addition, in the drawings used in the following descriptions, for convenience, characteristic portions may be enlarged for easy understanding of characteristics, and dimension ratios between components are not limiting to be being the same as actual dimension ratios. In addition, a material, a dimension, or the like exemplified in the following descriptions is an example, the present invention is not limited to the example, and the example may be appropriately modified and exemplified within a scope which does not depart from the gist.

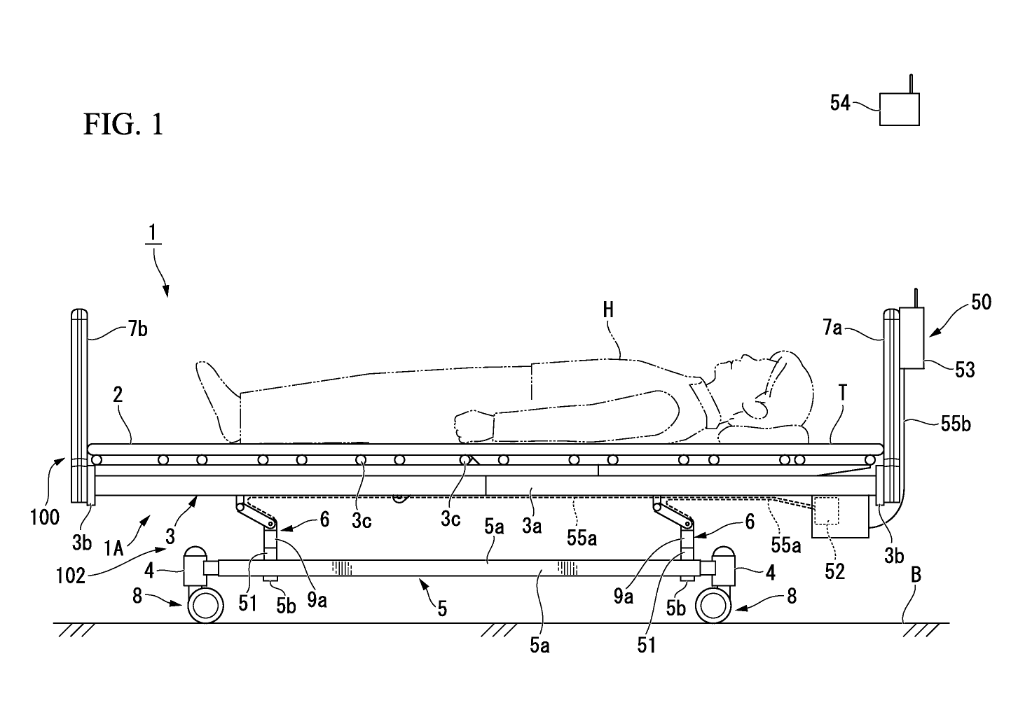

FIG. 1 is a side view showing an example of a bed 1 having a load detection function to which the present invention is applied, that is, is a side view of an example of a bed 1 into which a bed-load detector 50 is incorporated.

For example, the bed 1 having a load detection function includes a bed main body 1A which is installed on an installation surface B such as a floor surface, and has a function which detects a change of a load applied to the bed main body 1A using a load detector 50 attached to the bed main body 1A and detects a state of a user H on a bed surface T of the bed main body 1A.

Moreover, in the following descriptions, the installation surface B and the bed surface T of the bed main body 1A shown in FIG. 1 are horizontal surfaces (surfaces orthogonal to the gravity direction), in a state where the user H sleeps on the bed surface T of the bed main body 1A in a supine position, a head side of the user H is defined as a “front side of the bed main body 1A”, a foot side of the user H is defined as a “rear side of the bed main body 1A”, a right side of the user H is defined as a “right side of the bed main body 1A”, and a left side of the user H is defined as a “left side of the main body 1A”.

Specifically, the bed main body 1A is configured so as to approximately include a bed surface-forming portion 100 which forms the bed surface T, a leg portion 4 which comes into contact with the installation surface B on which the bed main body 1A is to be installed, and a connection-support portion 102 which connects the bed surface-forming portion 100 and the leg portion 4 so that the bed surface-forming portion 100 is positioned above the installation surface B and transmits a load from the bed surface-forming portion 100 to the leg portion 4.

Here, in an example shown in FIG. 1, the bed surface-forming portion 100 is configured of a bed plate 2 and an upper frame 3 which supports the bed plate 2. Moreover, the connection-support portion 102 includes a lower frame 5, and a lifting-lowering link mechanism 6 which connects the upper frame 3 and the lower frame 5 and lifts and lowers the bed plate 2 along with the upper frame 3.

The bed plate 2 is formed of a rectangular flat plate having a length and a width sufficient for sleeping of the user H.

For example, in the bed main body 1A, a mat, a mattress, or the like is laid on the bed plate 2, and in this state, the user H can be on the mat or the like (in addition, FIG. 1 shows a state where the user H directly lies on the upper surface (bed surface T) of the bed plate 2 horizontally).

The upper frame 3 has a structure (a frame structure) in which a pair of right and left pipes 3a which extends in a length direction (a longitudinal direction of the bed main body 1A) of the bed plate 2 and a pair of front and rear pipes 3b which extends in a width direction (a lateral direction of the bed main body 1A) of the bed plate 2 are connected to each other so as to be formed in a frame shape as a whole, and in a state where a plurality of pipes 3c extending in a width direction (the lateral direction of the bed main body 1A) of the bed plate 2 are arranged in the length direction (the longitudinal direction of the bed main body 1A) of the bed plate 2, the pair of right and left pipes 3a are connected to each other.

In addition, the bed plate 2 is attached in a state where the bed plate 2 is fixed to the upper portions of the plurality of pipes 3c. Moreover, a head plate 7a and a foot plate 7b are attached to the pair of front and rear pipes 3b configuring the upper frame 3 in a state where the plates 7a and 7b are erected upward in a vertical direction.

Four leg portions 4 are disposed at four corners (left front side, right front side, left rear side, and right rear side) of the bed main body 1A having a symmetrical positional relationship to each other. Moreover, a caster mechanism 8 for easily moving the bed main body 1A which is a heavy load is provided in each of four leg portions 4. A configuration of the caster mechanism 8 is not particularly limited, and a well-known configuration may be used. In addition, in some cases, the leg portion 4 may not include the caster mechanism.

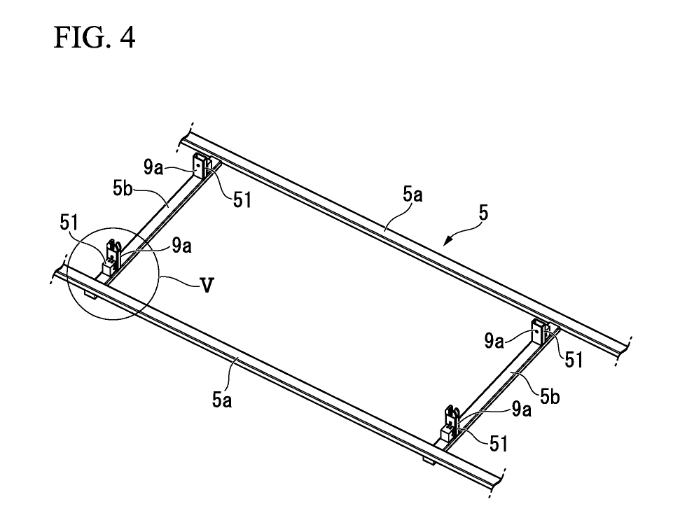

The lower frame 5 is formed in a planar frame structure as a whole by combining and connecting at least four square pipe-shaped pipes in a frame shape. That is, the lower frame 5 is configured of a pair of right and left pipes 5a which extends in the longitudinal direction of the bed main body 1A, and a pair of front and rear pipes 5b which extends in the lateral direction of the bed main body 1A, and both ends of the pair of front and rear pipes 5b are joined to locations close to both ends of the pair of right and left pipes 5a (refer to FIG. 4). Moreover, the leg portion 4 (caster mechanism 8) is provided on both end portions of the pair of right and left pipes 5a configuring the lower frame 5. In addition, in this example, each of the pipes 5b of the lower frame 5 corresponds to the configuration member of the installation surface side described in the aspect of (1).

The pair of lifting-lowering link mechanisms 6 in the above-described connection-support portion 102 is disposed so as to be arranged on a front side and a rear side of the bed main body 1A. Moreover, the front and rear lifting-lowering mechanisms 6 have substantially the same structure as each other except that the attachment positions are different from each other. In addition, each of the front and rear lifting-lowering link mechanism 6 has a bilaterally symmetrical structure with respect to the right side and the left side of the bed main body 1A.

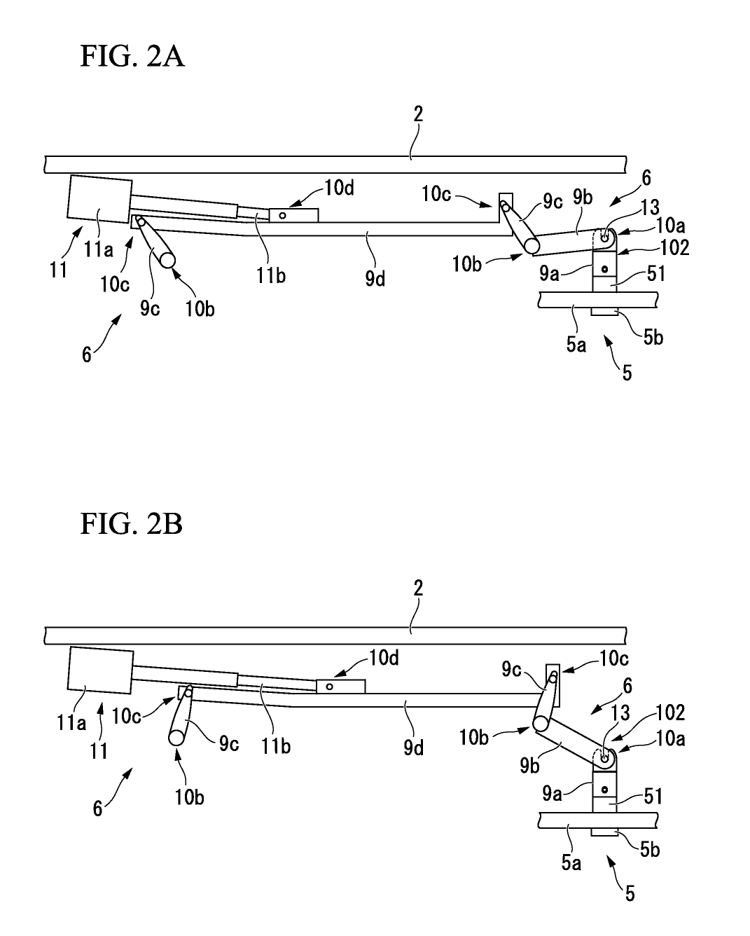

Accordingly, for example, as shown in FIGS. 2A and 2B, the front and rear lifting-lowering link mechanism 6 is collectively described if necessary.

Moreover, as an example of the lifting-lowering mechanism for lifting and lowering the bed plate 2, a swing lifting-lowering type lifting-lowering link mechanism 6 is shown. However, as the lifting-lowering mechanism, other link mechanisms, a pantograph type, a vertical lifting-lowering type, or the like may be used. Accordingly, as long as a spindle (pin) 13 having a substantially horizontal axial line described below is provided on an intermediate or end portions of the lifting-lowering mechanism as a member to which a load from the bed plate 2 is applied, similarly to a case where the lifting-lowering mechanism is configured of the swing lifting-lowering type lifting-lowering mechanism 6, the present invention can be applied to any type of lifting-lowering mechanism.

FIG. 2A is a side view of a main portion of the bed main body 1A showing a state where the bed plate 2 has been lowered along with the upper frame (not shown) by the lifting-lowering link mechanism 6. Meanwhile, FIG. 2B is a side view of a main portion of the bed main body 1A showing a state where the bed plate 2 has been lifted along with the upper frame (not shown) by the lifting-lowering link mechanism 6.

Specifically, as shown in FIGS. 2A and 2B, the lifting-lowering link mechanism 6 includes a pair of first right and left connection arms 9a, a pair of second right and left connection arms 9b, and a pair of third right and left connection arms 9c which are connected to each other between the upper arm 3 and the lower frame 5.

Among these, the first connection arms 9a are attached in a state where lower end portions of the arms 9a are fixed to the pair of front and rear pipes 5b configuring the lower frame 5. Moreover, the first connection arm 9a is formed in a hollow cylindrical shape, for example, a square tube shape, and corresponds to the hollow cylindrical support body (cylindrical support body) described in the aspect of (5), and includes a hollow portion inside the first connection arm along a vertical direction. Meanwhile, a lower end portion of the second connection arm 9b is rotatably attached to an upper end portion of the first connection arm 9a via a first hinge portion 10a. In addition, a lower end portion of the third connection arm 9c is rotatably attached to an upper end portion of the second connection arm 9b via a second hinge portion 10b.

Moreover, the lifting-lowering link mechanism 6 includes a pair of fourth right and left connection arms 9d which connects the third front and rear connection arms 9c. Moreover, each of upper ends of the third front and rear connection arms 9c is rotatably attached to the fourth connection arm 9d via a third hinge portion 10c.

Moreover, the lifting-lowering link mechanism 6 includes an actuator (driving mechanism) 11 for lifting and lowering the bed plate 2 along with the upper frame (not shown). The actuator 11 moves (expands and contracts) a piston 11b from a cylinder 11a in a front-rear direction using electricity. Here, the cylinder 11a is attached in a state where the cylinder 11a is fixed to the upper frame 5 (not shown in FIGS. 2A and 2B). Meanwhile, a tip portion of the piston 11b is rotatably attached to the fourth connection arm 9d via the fourth hinge portion 10d. Moreover, the actuator 11 is provided on only one of the right side and the left side of the bed main body 1A.

In addition, the piston 11b moves (is expanded) toward the front side by driving of the actuator 11, and accordingly, the lifting-lowering link mechanism 6 moves from the state where the bed plate 2 has been lowered along with the upper frame (not shown) as shown in FIG. 2A to the state where the bed plate 2 has been lifted along with the upper frame (not shown) as shown in FIG. 2B while the first to fourth connection arms 9a to 9d cooperate with one another. Conversely, the piston 11b moves (is contracted) toward the rear side by driving of the actuator 11, and accordingly, the lifting-lowering link mechanism 6 moves from the state where the bed plate 2 has been lifted along with the upper frame (not shown) as shown in FIG. 2B to the state where the bed plate 2 has been lowered along with the upper frame (not shown) as shown in FIG. 2A while the first to fourth connection arms 9a to 9d cooperate with one another. Accordingly, a height of the bed plate 2 can be adjusted while the bed plate 2 is lifted and lowered along with the upper frame (not shown). In addition, a load from the bed plate 2 is applied to the pin (spindle) 13 having a substantially horizontal axial line of the first hinge portion 10a in the lifting-lowering link mechanism 6.

As shown in FIG. 1, the load detector 50 includes a load cell 51 which measures strain generated when a load is applied to the bed main body 1A, and in the present example, as shown in FIG. 1, in addition to the load cell 51, the load detector 50 includes a calculation unit 52 which calculates a state of the user H on the bed surface T of the bed main body 1A based on a load signal output from the load cell 51, a transmission unit 53 which remotely transmits results calculated by the calculation unit 52, and a reception unit 54 which receives the signal transmitted from the transmission unit 53.

Moreover, the load cell 51 and the calculation unit 52 are connected to each other by a wire 55a, and the calculation unit 52 and the transmission unit 53 are connected to each other by a wire 55b. Meanwhile, transmission and reception can be performed between the transmission unit 53 and the reception unit 54 by wireless communication (radio waves).

However, in the bed 1 having a load detection function to which the bed-load detector of the present invention is applied, the load cell 51 is incorporated into a site which is positioned at any location on a load transmission path extending from the bed surface-forming portion 100 to the leg portion 4 via the connection-support portion 102, and at which a load from the bed surface-forming portion side 100 is received and the load is transmitted to the installation surface B side.

In addition, particularly, in the case of the example of FIG. 1, in the load transmission path, the load cell 51 is incorporated into the lifting-lowering link mechanism 6 of the connection-support portion 102. Accordingly, here, first, as described above, the case where the load cell 51 is incorporated into the lifting-lowering link mechanism 6 will be described in more detail.

Specifically, as shown in FIGS. 2A and 2B, the load cell 51 is attached to each of the first hinge portions 10a which are disposed at four corner (the left front side, the right front side, the left rear side, and the right rear side) having a symmetrical positional relationship to each other, in the first to fourth hinge portions 10a to 10d configuring the lifting-lowering link mechanism 6 (a total of four load cells are attached to the first hinge portions 10).

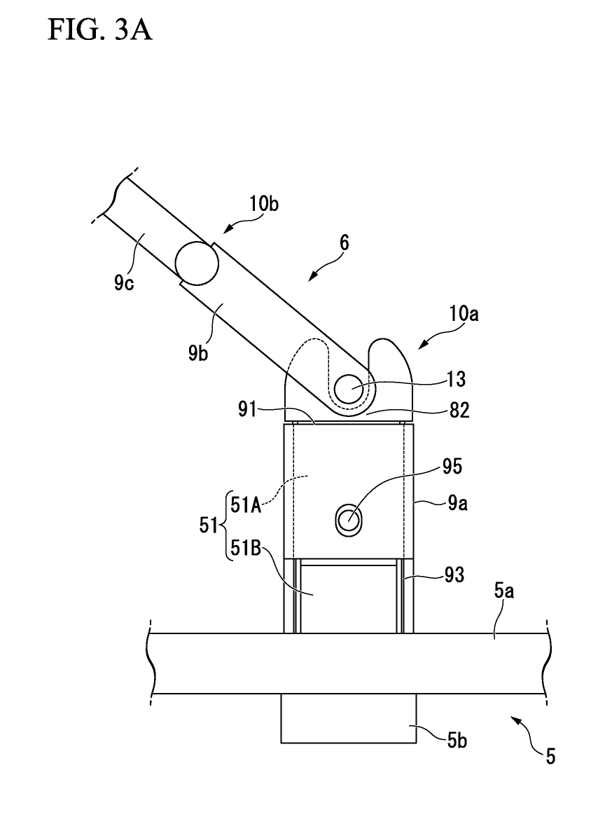

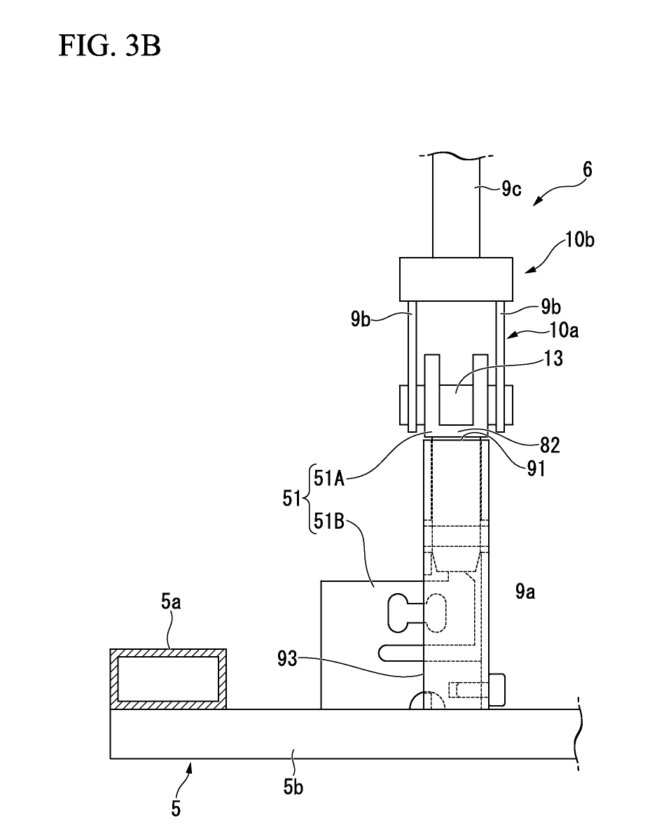

Moreover, the four load cells 51 have substantially the same structure as each other except that the attachment positions are different from each other. Accordingly, for example, as shown in FIGS. 3A and 3B, the four load cells 51 are collectively described.

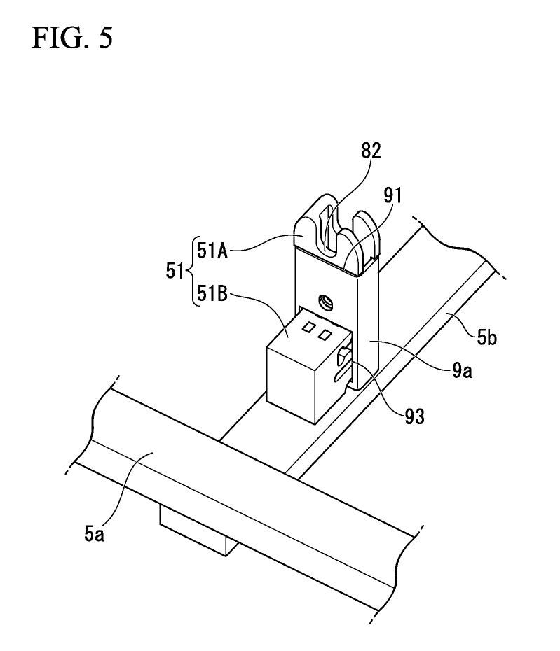

Here, FIG. 3A is an enlarged side view of main portions of the lifting-lowering link mechanism 6 into which the load cell 51 is incorporated, and the lower frame 5. Meanwhile, FIG. 3B is an enlarged front view of main portions of the lifting-lowering link mechanism 6 into which the load cell 51 is incorporated, and the lower frame 5. In addition, FIG. 4 shows a relationship between the lower frame 5 and the load cells 51 in the bed main body 1A shown in FIG. 1, and FIG. 5 shows a main portion of FIG. 4.

As shown in FIGS. 3A, 3B, 4, and 5, the first connection arm (one connection arm; cylindrical support body) 9a is perpendicularly erected to the pipe 5b of the lower frame 5 so as to be extended above from the pipe 5b. The first connection arm 9a is the hollow cylindrical support body (cylindrical support body) described in the fifth aspect, and in the present embodiment, the first connection arm 9a is formed in a hollow square pipe shape (square pipe shape) having a rectangular shape in a horizontal section and is fixed to the pipe 5b using arbitrary fixing means such as welding or brazing. In addition, the upper end of the square tube-shaped connection arm 9a is open (upper opening end 91), and for example, a side wall of a lower portion of the square tube-shaped connection arm 9a is cut in a rectangular shape, and a side opening portion 93 is formed. Moreover, here, the side opening portion 93 is open in a direction (a lateral direction of the bed main body 1A) along the length direction of the pipe 5b of the lower frame 5.

Meanwhile, as described in detail below again, the load cell 51 is configured to include a load-receiving member 51A which receives a load from the bed surface-forming portion 100 side, and a base body 51B which is mechanically (structurally) separated from the load-receiving member 51A and to which the load from the load-receiving member 51A is applied. In addition, the lower portion of the load-receiving member 51A in the load cell 51 is inserted from the upper opening end 91 of the square tube-shaped connection arm (cylindrical support body) 9a into the inside of the upper portion of the square tube-shaped connection arm 9a, and the base body 51B in the load cell 51 is inserted from the side opening portion 93 of the side wall of the lower portion of the square tube-shaped connection arm 9a into the inside of the lower portion of the square tube-shaped connection arm 9a.

Moreover, the above-described first hinge portion 10a has a structure in which the first hinge portion 10a is pivoted in a state where the pin (spindle) 13 provided in the second connection arm (another connection arm) 9b engages with a bearing portion 82 formed in the load-receiving member 51A of the load cell 51 described in detail below, and the second connection arm 9b is rotatably supported.

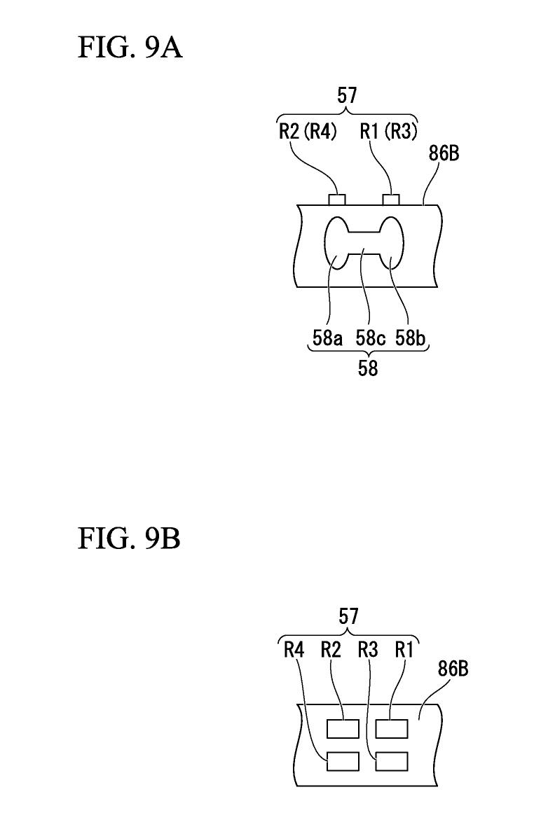

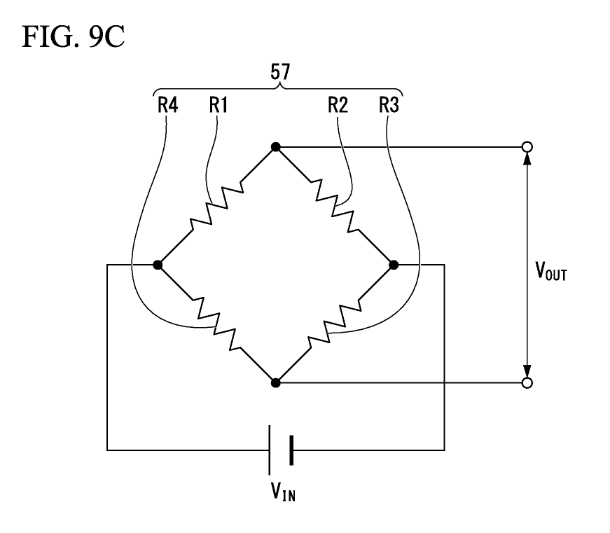

Basically, as described above, the load cell 51 includes a load-receiving member 51A which receives a load from the bed surface-forming portion 100 side, and a base body 51B which is mechanically (structurally) separated from the load-receiving member 51A and to which a load from the load-receiving member 51A is applied, and a strain sensor 57 (refer to FIGS. 9A to 9C) which detects strain of the base body 51A is attached to the base body 51A.

Moreover, specific examples of the load-receiving member 51A and the base body 51B will be described.

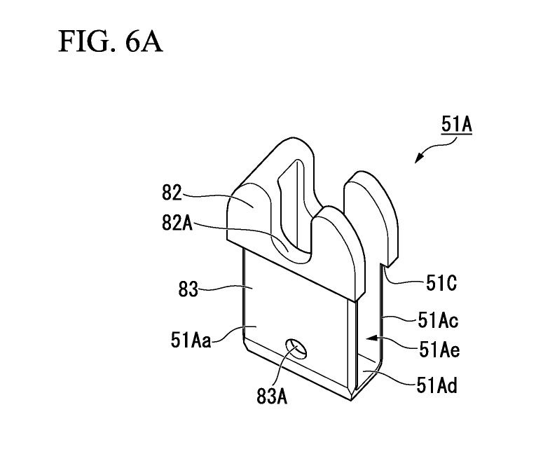

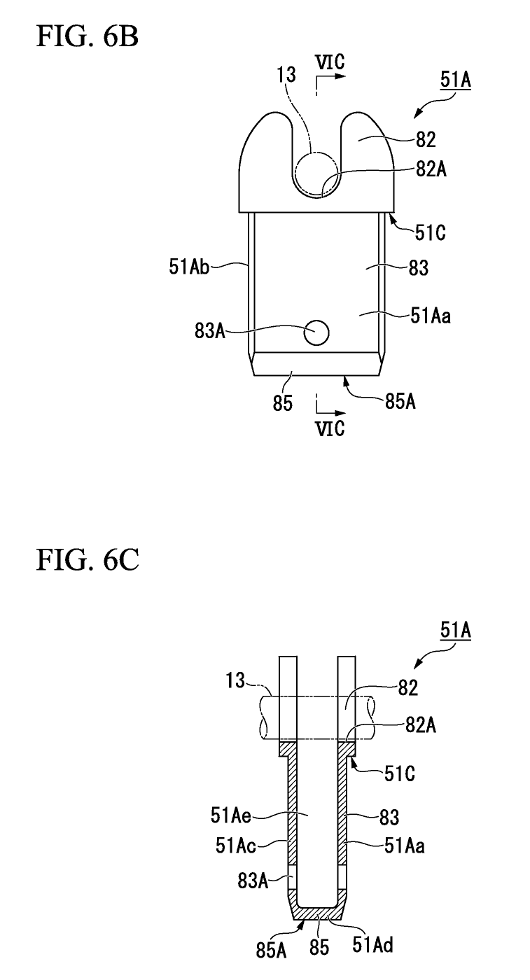

FIGS. 6A to 6C show a first example of the load-receiving member 51A. Moreover, the load-receiving member 51A of the first example has a shape similar to the load-receiving member 51A of the load cell 51 shown in FIG. 3A.

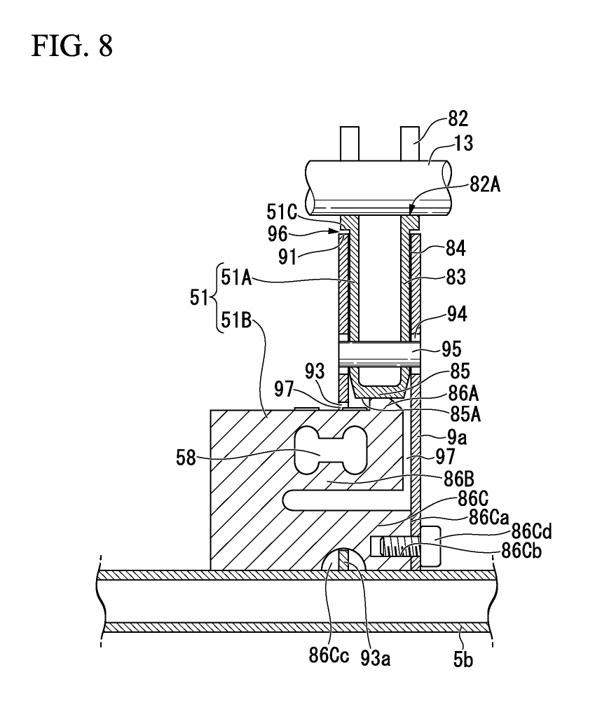

In FIGS. 6A to 6C, the bearing portion 82 which is open in a U shape upward is provided on the upper portion of the load-receiving member 51A. That is, the bearing portion 82 bifurcates upward, and the bifurcated bottom surface is formed in a concave curved surface and becomes a bearing surface 82A which rotatably receives the above-described spindle (pin) 13. Meanwhile, the lower portion of the load-receiving member 51A extends downward from the bearing portion 82, and becomes an insertion portion 83 which is inserted from the upper opening end 91 of the above-described square tube-shaped connection arm (cylindrical support body) 9a into the connection arm 9a. Similarly to the shape in the horizontal section of the above-described square tube-shaped connection arm 9a, an outer shape in the horizontal section of the insertion portion 83 is formed in a rectangular shape. However, dimensions of the outer shape (rectangle) in the horizontal section of the insertion portion 83 are determined so as to be slightly smaller than the inner dimensions in the horizontal section of the square tube-shaped connection arm 9a, and accordingly, as described again below, a gap 84 is formed between an outer surface of the insertion portion 83 and an inner surface of the square tube-shaped connection arm 9a (refer to FIG. 8).

In addition, similarly to the outer shape in the horizontal section of the insertion portion 83, the outer shape in the horizontal section of the bottom portion of the bearing portion 82 in the load-receiving member 51 is also formed in a rectangular shape. However, the outer shape of the bottom portion of the bearing portion 82 is larger than the outer shape of the insertion portion 83, and the dimensions of the outer shape of the bottom portion are approximately similar to the outer dimensions in the horizontal section of the square tube-shaped connection arm 9a. Accordingly, a step portion 51C is formed between an outer edge of the bottom portion of the bearing portion 82 and an outer surface of the insertion portion 83. Moreover, a shaft hole 83A which penetrates in a horizontal direction parallel to an axial direction of the above-described spindle (pin) 13 is formed in the insertion portion 83.

In addition, a contact portion 85, which protrudes in a trapezoidal shape in a vertically downward direction so as to come into contact with the base body 51B, is formed on the lower end of the insertion portion 83. In the present example, the lower end surface (abutment surface) 85A of the contact portion 85 is a horizontally rectangular surface which extends in a direction orthogonal to the axial direction of the above-described spindle (pin) 13.

In addition, the entire load-receiving member 51A has a shape which is notched from one side and the upper side other than side wall sites 51Aa, 51Ab, 51Ac corresponding to three side surfaces among four perpendicular side surfaces and a bottom wall site (a portion at which the contact portion is positioned) 51Ad corresponding to a horizontal bottom surface, that is, a shape in which a portion surrounded by three side wall sites 51Aa, 51Ab, and 51Ac, and the bottom site 51Ad becomes a space 51Ae. This shape is adopted only for a reduction in weight and reduction in cost of materials of the load-receiving member 51A. Accordingly, a solid shape in which the space 51Ae is not present may be adopted.

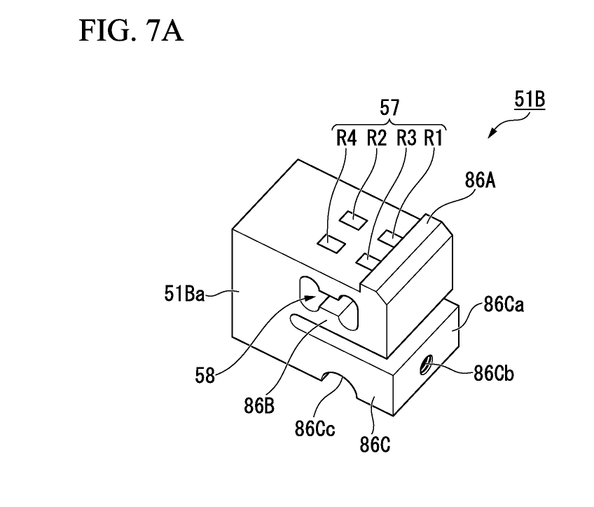

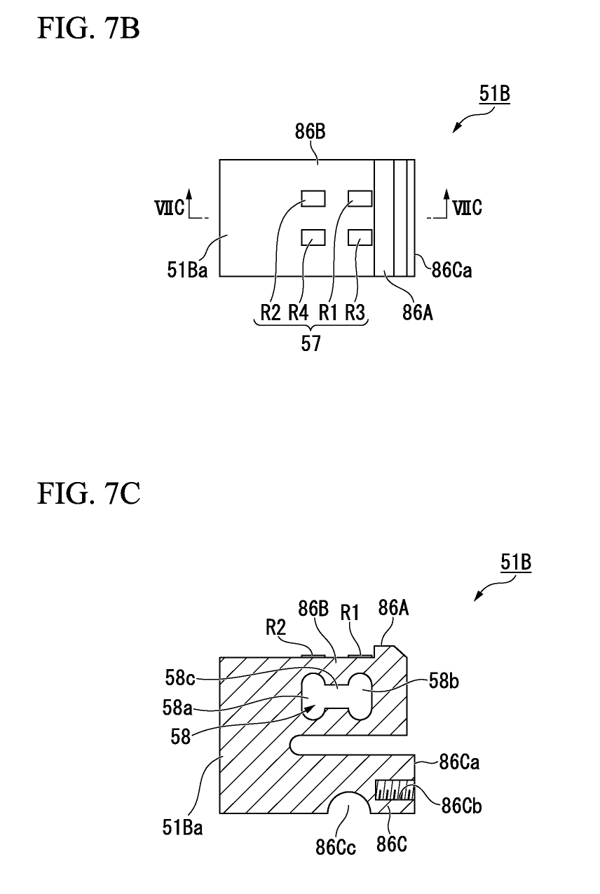

FIGS. 7A to 7C show a first example of the base body 51B of the load cell 51. In addition, the base body 51B of the first example has the same shape as that of the base body 51B of the load cell 51 shown in FIGS. 3A, 3B, and 5.

Basically, the base body 51B includes an action portion 86A with which the contact portion 85 of the load-receiving member 51A comes into contact and on which a load from the load-receiving member 51A acts, an operating portion 86B which is strain-deformed by the load applied to the action portion 86A, and an attachment portion 86C which is continuous with the operating portion 86B and is fixed to a configuration member (in the case of the present embodiment, the pipe 5b of the lower frame 5, or the square tube-shaped connection arm 9a on the pipe 5b) of the installation surface B side in the bed main body 1A. In addition, the strain sensor 57 is attached to the operating portion 86B.

Here, the base body 51B corresponds to a so-called strain element, and in the present example, a cantilever type configuration is applied to the base body 51B.

Specifically, as shown in FIGS. 7A to 7C, the operating portion 86B of the base body 51B has a shape which has one side (left side in FIGS. 7A to 7C) portion in a horizontal direction as a base portion 51Ba and extends in a cantilever shape along a horizontal direction toward the other side (right side in FIGS. 7A to 7C) in the horizontal direction from the upper portion of the base portion 51Ba. That is, the operating portion 86B becomes a cantilever portion. In addition, an upper surface on an extended end side in the operating portion 86B protrudes upward, and the protrusion portion becomes the action portion 86A, that is, the action portion 86A with which the contact portion 85 of the load-receiving member 51A comes into contact from above. Moreover, the attachment portion 86C extends in a direction parallel to the direction in which the operating portion 86B extends from the lower portion of the above-described base portion 51Ba, a tip surface 86Ca of the attachment portion 86C becomes a perpendicular surface, and a screw hole 86Cb is formed along the horizontal direction from the tip surface 86Ca. In addition, a concave portion 86Cc for straddling a portion 93a remaining on the lower side of the side opening portion 93 in the above-described square tube-shaped connection arm (cylindrical support body) 9a is formed on the lower surface side of the attachment portion 86C.

Moreover, in the present example, the action portion 86A is configured of a protrusion portion protruding upward. However, the action portion 86A is not necessarily a protrusion portion, and may be a planar portion on an upper surface of an extended end portion of the operating portion (cantilever portion) 86B. In addition, when the action portion 86A is configured of a protrusion portion, the upper surface of the action portion may be a horizontal plane surface or may be a curved surface which is convex-curved upward. In addition, in contrast with the protrusion portion, the action portion 86A may be a concave portion which is formed so as to be recessed downward on the upper surface of the extended end portion of the operating portion (cantilever portion) 86B. In any case, importantly, a surface which receives the lower end surface (abutment surface) 85A of the contact portion 85 in the above-described load-receiving member 51A may be provided.

Here, a hole portion 58 for configuring a Roberval mechanism is provided on the operating portion (cantilever portion) 86B. As shown in FIGS. 7C and 9A, the hole portion 58 penetrates the operating portion (cantilever portion) 86B in a width direction, and is formed to include a pair of round holes 58a and 58b which is horizontally arranged along a length direction (a direction which horizontally extends; a length direction of the cantilever) of the operating portion 86B, and a connection hole 58c which connects centers of the pair of round holes 58a and 58b.

Moreover, for example, the strain sensor 57 adheres to the upper surface of the operating portion (cantilever portion) 86B. The strain sensor 57 detects a change of strain by a change of an electrical resistance according to the strain generated in the operating portion (cantilever portion) 86B. In the present example, the strain sensor 57 includes four strain gauges (strain sensitive resistors) R1, R2, R3, and R4, and as shown in FIGS. 9A and 9B, in the strain gauges R1, R2, R3, and R4, each of the pair of strain gauges R1 and R3 and the pair of strain gauges R2 and R4 is arranged immediately on positions at which the pair of round holes 58a and 58b of the operating portion (cantilever portion) 86B is formed in the width direction of the operating portion (cantilever portion) 86B.

The four strain gauges R1, R2, R3, and R4 configure a Wheatstone bridge circuit as shown in FIG. 9C, and among these, R1 and R3 become compression side strain gauges, and R2 and R4 become tension side strain gauges. In addition, in the Wheatstone bridge circuit, it is possible to output an output voltage VOUT (load signal) corresponding to a magnitude of strain generated in the operating portion (cantilever portion) 86B with respect to an input voltage VIN (constant).

Moreover, the strain sensor 57 may be configured of at least two or three strain gauges (strain sensitive resistors). In this case, among the strain gauges R1, R2, R3, and R4 configuring the Wheatstone bridge circuit shown in FIG. 9C, one, two, or the three strain gauges may be replaced by dummy resistances which are resistors which do not have strain sensitivity.

In addition, a material of the load-receiving member 51A of the load cell 51 and a material of the base body 51B serving as the strain element are not particularly limited. It is possible to select a suitable material as a configuration material according to required characteristics of each of the load-receiving member 51A and the base body 51B, for example, preferable workability, yield strength, elongation, abrasion resistance, or the like of each portion. That is, since the load-receiving member 51A is a member which receives a load from the spindle (pin) 13 and applies a force generated by the load to the base body 51B and is a portion which is not directly involved in generation of strain necessary for detecting a load, improved workability is required for the load-receiving member 51A such that the load-receiving member 51A can be easily processed to a suitable shape so as to receive a load from the spindle (pin) 13, and elongation or yield strength need not be considered. Meanwhile, since the base body 51B is a member which includes the operating portion (cantilever portion) 86B which is bent and deformed by a force generated from the spindle (pin) 13 and the base body 51B serves as a portion of a structure which is fixed to and supported by the member of the bed main body, it is preferable that yield strength increase and elongation decrease.

Specifically, a metal such as aluminum alloy, iron, steel, or stainless steel, or a resin such as an engineering plastic may be used for a material of the load-receiving member 51A of the load cell 51 and a material of the base body 51B serving as a strain element. In addition, a material of the load-receiving member 51A and a material of the base body 51B may not be the same as each other, and for example, preferably, the materials are selected from the above-described viewpoints, or from the viewpoints of lightness or economic efficiency, that is, a resin such as an ABS resin or a polycarbonate resin may be used for the load-receiving member 51A, and light alloy such as aluminum alloy, titanium alloy, or magnesium alloy, or a metal such as iron, carbon steel, or stainless steel may be used for the material of the base body 51B.

A state in which the above-described load cell 51 (the first example of the load-receiving member 51A and the first example of the base body 51B) is incorporated into the square tube-shaped connection arm (cylindrical support body) 9a on the pipe (the structural member of the installation surface side) 5b of the lower frame 5 in the bed main body 1A is shown in FIGS. 3A, 3B, 5, and 8. Accordingly, the state in which the load cell 51 is incorporated into the connection arm 9a will be described with reference to the drawings (mainly, FIG. 8).

The base body 51B of the load cell 51 is placed on the upper surface of the pipe 5b so that an extension direction of the operating portion (cantilever portion) 86B from the base portion 51Ba is positioned along the length direction of the pipe 5b on the upper surface of the pipe 5b of the lower frame 5. In addition, a portion of the tip portion side in the base body 51B (a portion of a side on which the protruding action portion 86A is positioned on the extended end side of the operating portion (cantilever portion) 86B) is inserted into the square tube-shaped connection arm 9a from the side opening portion 93 of the square tube-shaped connection arm 9a, and the tip surface 86Ca of the attachment portion 86C comes into contact with an inner wall surface of the square tube-shaped connection arm 9a opposing the tip surface 86Ca. Moreover, a screw 86Cd is inserted into the screw hole 86Cb from the outside of the square tube-shaped connection arm 9a and is screwed. Accordingly, the attachment portion 86C is fixed to the square tube-shaped connection arm 9a. Meanwhile, a portion of a base end side in the base body 51B (a portion of the base portion 51Ba side) is positioned outside the side opening portion 93 of the square tube-shaped connection arm 9a. Accordingly, the entire base body 51B is not inserted into the square tube-shaped connection arm 9a, and only a portion (a portion in which at least the protruding action portion 86A is positioned) of the entire base body 51B is inserted into the square tube-shaped connection arm 9a.

In addition, here, in the base body 51B, gaps 97 are secured between the action portion 86A and the operating portion (cantilever portion) 86B, and the inner wall surface and the edge portion of the side opening portion 93 of the square tube-shaped connection arm 9a so that the action portion 86A and the operating portion (cantilever portion) 86B do not come into contact with the inner wall surface and the edge portion of the side opening portion 93 of the square tube-shaped connection arm 9a.

Meanwhile, the insertion portion 83 of the lower portion of the load-receiving member 51A of the load cell 51 is vertically inserted from the upper opening end 91 of the square tube-shaped connection arm 9a into the square tube-shaped connection arm 9a. Moreover, the lower end surface (abutment surface) 85A of the contact portion 85 of the insertion portion 83 comes into contact with the upper surface of the protruding action portion 86A of the base body 51B. Here, a gap 84 exists between the outer surface of the load-receiving member 51A and the inner wall surface of the square tube-shaped connection arm 9a.

In addition, an auxiliary pin 95 is inserted into and fixed to the shaft hole 83A of the insertion portion 83 of the load-receiving member 51A so that both end portions of the auxiliary pin 95 protrude toward both sides of the insertion portion 83, and both end portions of the auxiliary pin 95 are inserted into long holes 94 which are formed on both side walls of the square tube-shaped connection arm 9a. Here, the auxiliary pin 95 is disposed to regulate the position of the load-receiving member 51A, and can freely move in a direction which is perpendicular to the long holes 94 of the square tube-shaped connection arm 9a with a small friction resistance. In this way, since the auxiliary pin 95 is provided, deviation of the position of the load-receiving member 51A is prevented, and the load-receiving member 51A can be slightly inclined about the auxiliary pin 95. Moreover, arbitrary means for fixing the auxiliary pin 95 to the insertion portion 83 of the load-receiving member 51A may be adopted. For example, the auxiliary pin 95 may be configured of a screw rod, the shaft hole 83A may be configured of a female screw hole, and the auxiliary pin 95 may be screwed into the shaft hole 83A, or the auxiliary pin 95 may be fixed to the shaft hole 83A using welding, brazing, or the like.

The bearing portion 82 of the upper portion of the load-receiving member 51A of the load cell 51 is positioned above the upper opening end 91 of the square tube-shaped connection arm 9a. That is, the step portion 51C which is positioned at a boundary between the bearing portion 82 and the insertion portion 83 is positioned slightly above the upper opening end 91 of the square tube-shaped connection arm 9a, and a space 96 exists between the upper opening end 91 and the step portion 51C.

In this way, the load-receiving member 51A protrudes above from the upper opening end 91 of the square tube-shaped connection arm 9a. In addition, the spindle (pin) 13 of the lifting-lowering link mechanism 6 in the bed main body 1A is inserted into the bearing portion 82 having a U shape in the load-receiving member 51A, and a load from the bed surface-forming portion 100 side in the bed main body 1A is applied to the bearing portion 82 via the spindle (pin) 13, particularly to the bearing surface 82A.

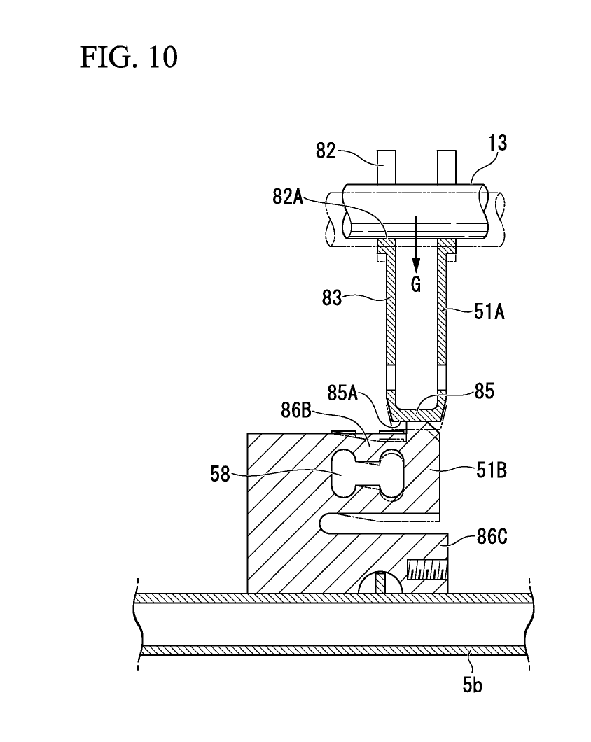

FIG. 10 shows a state when a load from the bed surface-forming portion 100 side in the bed main body 1A is applied to the load cell 51, which is obtained by combining the load-receiving member 51A of the above-described first example and the base body 51B of the above-described first example, via the spindle (pin) 13, and in this case, the state of the load cell 51 will be described below. In addition, in FIG. 10, a solid line shows a state before a load G is applied, and a chain line shows a state when the load G is applied. However, in FIG. 10, the state when the load G is applied is exaggeratively shown. In addition, in FIG. 10, for simplicity, the square tube-shaped connection arm (cylindrical support body) 9a is omitted. Accordingly, with respect to a description of a relationship between the square tube-shaped connection arm 9a and the load cell 51, refer to FIG. 8.

When the load G in the vertically downward direction is applied from the bed surface-forming portion 100 of the bed plate 2 or the like to the load-receiving member 51A of the load cell 51, particularly, to the bearing surface 82A of the bearing portion 82 via the spindle (pin) 13 of the link mechanism 6, the contact portion 85 of the load-receiving member 51A presses the protruding action portion 86A in the base body 51B of the load cell 51. That is, the load G applied to the load-receiving member 51A is transmitted to the action portion 86A of the base body 51B. Accordingly, the tip side of the operating portion (cantilever portion) 86B continuous with the action portion 86A is pressed, and strain is generated in the operating portion 86B. In this case, the strain sensor 57 detects a change of a resistance according to a magnitude of the strain generated in the operating portion 86B, and outputs a strain signal corresponding to the magnitude of the strain generated in the operating portion 86B, that is, a signal corresponding to the change of the load. In addition, it is possible to detect the change of the load applied to the bed surface-forming portion 100 of the bed plate 2 or the like using the load detector 50 including the load cell 51.

Here, when a load is applied from the spindle (pin) 13 to the load-receiving member 51A, the load-receiving member 51A is pressed downward. However, since the space 96 (refer to FIG. 8) exists between the step portion 51C and the upper opening end 91 of the square tube-shaped connection arm 9a in the load-receiving member 51A, the gap 84 (refer to FIG. 8) exists between the outer surface of the insertion portion 83 and the square tube-shaped connection arm 9a in the load-receiving member 51A, and the auxiliary pin 95 inserted into the insertion portion 83 can move in the vertical direction due to the long holes 94 of the side walls of the square tube-shaped connection arm 9a, the load-receiving member 51A can be lowered according to a load applied to the load-receiving member 51A. That is, when the load-receiving member 51A receives the load G in the vertically downward direction, the load-receiving member 51A is lowered in a state where a friction resistance is not substantially generated between the load-receiving member 51A and the square tube-shaped connection arm 9a and can transmit the load to the base body 51B. In addition, since the outer surfaces of the action portion 86A and the operating portion (cantilever portion) 86B of the base body 51B are separated from the inner wall surface of the square tube-shaped connection arm 9a or the edge of the side opening portion 93 due to the gap 97 (refer to FIG. 8), the operating portion (cantilever portion) 86B is deformed (bent) according to a load, strain is generated, the strain is detected by the strain sensor 57 as described above, and it is possible to correctly detect the load G in the vertically downward direction.

Here, according to a state or a movement of a user of a bed, a biased load may be applied to the bed surface 3 of the bed main body 1A. Specifically, a user of a bed, a visitor, a health care worker, or the like may sit on an end portion of the bed surface 3, a user who lies on the bed surface 3 mainly has turned over toward an end portion side on the bed surface 3 or stands up on the bed surface 3, and when a load applied to the bed surface 3 is greatly biased (this state is referred to as an unbalanced load state), the entire bed main body 1A may be slightly distorted. In this case, according to the distortion, a twist (here, the twist means that a center axis is inclined with respect to an original center axis position of the spindle) or positional deviation (for example, a parallel movement in a horizontal surface) may occur in the spindle 13. In this case, a force in a horizontal direction or an inclination direction is applied to the bearing portion 82 of the load-receiving member 51A in the load cell 51 according to the inclination or the movement of the spindle 13. This means that with respect to an original load (force) in a vertical direction detected by the load cell 51, a component of a force in a direction different from the vertical direction is applied to the load-receiving member 51A of the load cell 51.

In addition, when it is assumed that the load-receiving member 51A of the load cell 51 and the base body 51B are integrally continuous with each other, in the unbalanced load state, not only is strain due to the force in the vertical direction generated in the operating portion (cantilever portion) 86B of the base body 51B but also strain due to the force in the horizontal direction or the inclination direction is superimposed on the strain due to the force in the vertical direction, and as a result, it is not possible to correctly detect the load in the vertical direction and there is a concern that detection accuracy of a load may be decreased.

However, in the case of the present invention, since the load-receiving member 51A of the load cell 51 and the base body 51B are mechanically and structurally separated from each other and only come into contact with each other in an up-down direction, only a component in a direction vertically downward from a load applied to the load-receiving member 51A is applied from the contact portion 85 of the load-receiving member 51A to the action portion 86A of the base body 51B. Accordingly, even when a load is applied to the bearing portion 82A in a state where a force in the horizontal direction or the inclination direction is superimposed on the force in the vertically downward direction in the unbalanced load state, only the component of the force in the vertically downward direction is applied to the base body 51B. As a result, strain is generated in the operating portion (cantilever portion) 86B of the base body 51B by only the component of the force in the vertically downward direction. Accordingly, even in the unbalanced load state in which the force in the horizontal direction or the inclination direction is applied, it is possible to correctly detect the load in the vertically downward direction without an affect from the force in the horizontal direction or the inclination direction.

In addition, when a biased load is applied to the bed surface-forming portion 100 of the bed main body 1A as described above, a side to which a load is not applied (or a side on which the load is smaller) in the bed main body 1A may rise up. In this case, when it is assumed that the load cell 51 is not separated from the load-receiving member 51A and the base body 51B and is integrally continuous with the load-receiving member 51A and the base body 51B, there is a concern that an upward force may be applied to the load cell 51. Accordingly, the load cell 51 detects the upward force as the load in the vertically upward direction (that is, the load which is negative with respect to the load in the vertically downward direction which is intended to be detected), and there is a concern that an error with respect to the original load detection may increase. However, in the case of the present invention, since the load cell 51 is separated from the load-receiving member 51A and the base body 51B in the up-down direction, even when the above-described load in the vertically upward direction (minus load) is applied to the load-receiving member 51A, the force is not transmitted to the base body 51B. Accordingly, the operating portion (cantilever portion) 86B of the base body 51B is not bent and it is possible to detect only the load in the vertically downward direction with high accuracy.

Here, preferably, the gap 84 (refer to FIG. 8) between the side surface of the load-receiving member 51A of the load cell 51 and the inner wall surface of the square tube-shaped connection arm (cylindrical support member) 9a is set so that the load-receiving member 51A is inclined by approximately ±1° to ±5° about the auxiliary pin 95. The specific gap 84 is changed according to a size of the load-receiving member 51A, a position of the auxiliary pin 95, or the like, and in general, may be approximately 0.1 mm to 10 mm.

In addition, in order to reliably perform the operation of transmitting only the force in the vertically downward direction from the contact portion 85 of the load-receiving member 51A as described above to the action portion 86A of the base body 51B, preferably, friction between the lower end surface (abutment surface) 85A of the contact portion 85 and the upper surface of the action portion 86A is decreased, and when the load-receiving member 51A is inclined, the base body 51B does not receive influence of the inclination.

Accordingly, first, in order to decrease the friction resistance between the lower end surface (abutment surface) 85A of the contact portion 85 and the upper surface of the action portion 86A, preferably, at least one of the surfaces is finished to a smooth surface (for example, a mirror surface). In addition, in some cases, surface processing for decreasing the friction resistance may be performed on at least one of the surfaces, or a low-friction (solid lubricant) film, for example, a fluororesin coating may be applied on at least one surface.

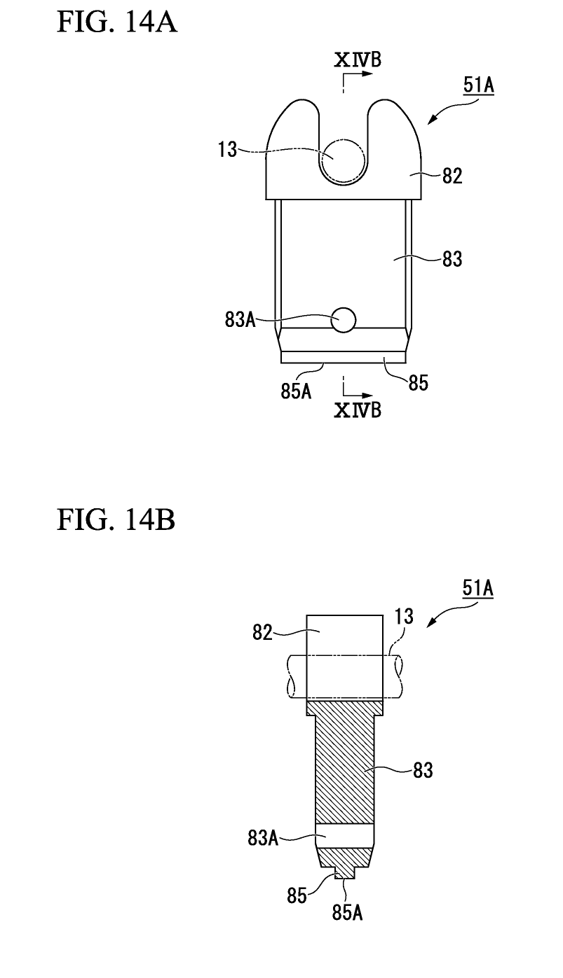

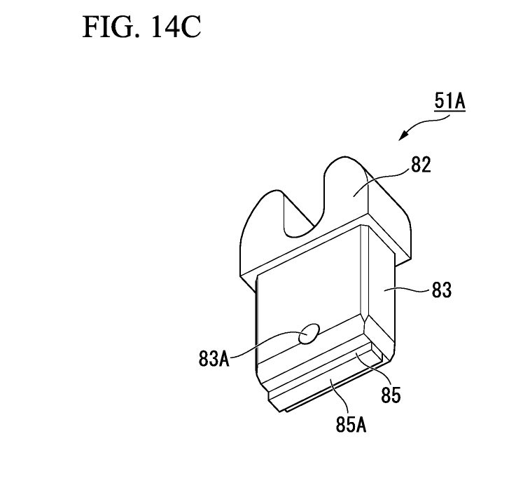

Moreover, secondarily, preferably, a contact area between the lower end surface (abutment surface) 85A of the contact portion 85 and the upper surface of the action portion 86A is decreased. Accordingly, for example, decreasing the area of the abutment surface 85A by changing the shape (shape which protrudes downward) of the contact portion 85 may be considered, or a contact state may be set to a line contact or a point contact rather than a surface contact. Some specific examples in which the shape (shape which protrudes downward) of the contact portion 85 is changed in this way so that the contact area between the contact portion 85 and the action portion 86A is decreased or the contact state is substantially changed from a surface contact to a point contact will be described in detail with reference to FIGS. 14A to 19B below.

In any case, the member (load-receiving member 51A) of the load cell 51 receiving a load and the member (base body 51B) in which strain is generated by a load are separate and independent from each other, the load-receiving member 51A and the base body 51B come into contact with each other in the up-down direction, and only the component in the vertically downward direction of a load which is received by the load-receiving member 51A is applied to the base body 51B. Therefore, any configuration may be adopted as long as the load-receiving member 51A and the base body 51B simply come into contact with each other in the up-down direction. Accordingly, degrees of freedom in shapes, dimensions, or attachment positions of the configuration members of the load cell 51 increase, and the load cell 51 can be incorporated into a bed on the market in a state where a design of the bed is not particularly changed.

In addition, in the above-described example, in the base body 51B of the load cell 51, only a portion including the action portion 86A is inserted into the square tube-shaped connection arm (cylindrical support body) 9a, and the remaining portion protrudes outside the square tube-shaped connection arm 9a. The reason why only a portion of the base body 51B is inserted into the square tube-shaped connection arm 9a in this way is as follows.

That is, a bending amount of the operating portion 86B increases even when the same load is applied as a length (a length from the action portion 86A to the base portion 51Ba) of the operating portion (cantilever portion) 86B of the base body 51B in the load cell 51 increases, and it is possible to detect a load with higher accuracy. Meanwhile, in a general bed main body 1A, in most cases, a width of the pipe 5b of the lower frame 5 is limited. Accordingly, in order to place the base body 51B on the pipe 5b having a relatively narrow width even when the length of the operating portion (cantilever portion) 86B increases, preferably, the length direction of the operating portion (cantilever portion) 86B is along the length direction of the pipe 5b. In addition, from the viewpoint of economic efficiency or the like, preferably, the dimensions (inner dimensions in the horizontal surface) of the square tube-shaped connection arm 9a in a general bed main body 1A are not increased.

As described above, if the length direction of the operating portion (cantilever portion) 86B is along the length direction of the pipe 5b, only a portion (a portion in which the action portion 86A is positioned) of the base body 51B is inserted into the square tube-shaped connection arm 9a, and the remaining portion protrudes outside the square tube-shaped connection arm 9a, even when the width of the pipe 5b is narrow and the dimensions of the square tube-shaped connection arm 9a in the horizontal surface are small, the length of the operating portion (cantilever portion) 86B increases, and it is possible to increase detection accuracy of the load. In other words, it is possible to increase the detection accuracy of a load by increasing a length of the operating portion (cantilever portion) 86B without limiting the width of the pipe which supports the base body 51B of the load cell 51.

In addition, as described above, only a portion (a portion in which the action portion 86A is positioned) of the base body 51B is inserted into the square tube-shaped connection arm 9a, the remaining portion protrudes outside the square tube-shaped connection arm 9a, and particularly, the attachment location of the strain sensor 57 in the operating portion (cantilever portion) 86B is set to the exterior surface of the square tube-shaped connection arm 9a (or, a location which is positioned inside the square tube-shaped connection arm 9a but is positioned so as to be close to the side opening portion 93). Accordingly, it is possible to easily replace the strain sensor 57 without disassembling the bed main body or removing the configuration members of the bed main body. In addition, in this case, it is not necessary to draw wires or cables connected from the strain sensor 57 to the outside around the inner portion of the square tube-shaped connection arm 9a. Accordingly, it is possible to easily hold wires or cables.

Hereinbefore, the state where one load cell 51 configured of the first example of the load-receiving member 51A and the first example of the base body 51B is incorporated into the square tube-shaped connection arm 9a positioned at one location on one pipe 5b of the lower frame 5 in the bed main body 1A is described. However, in the bed having a load detection function of the present invention, actually, preferably, the load cell 51 is incorporated into each of four corners (the left front side, the right front side, the left rear side, and the right rear side) of the bed main body 1A, or each of locations corresponding to the vicinities of the four corners, and changes of loads applied to four corners of the bed main body 1A are detected by a total of four load cells 51. In addition, preferably, load signals detected by the four load cells 51 are output to the calculation unit 52. Hereinafter, this will be described again with reference to FIG. 1.

The calculation unit 52 is configured of a computer which includes a ROM, a RAM, other memories, a CPU, or the like, and programs, numerical values, or the like required for calculating a state of the user H on the bed surface T of the bed main body 1A are stored in the calculation unit 52 in advance.

Moreover, in the calculation unit 52, the state of the user H on the bed surface T of the bed main body 1A is calculated based on load signals output from the four load cells 51, and the calculated results are output to the transmission unit 53.

For example, in the calculation unit 52, from the load signals output from the four load cells 51, when a total value of loads applied to the four load cells 51 is larger than a threshold value which is stored in advance, it is determined that the user H is on the bed surface T of the bed main body 1A, and the calculated results are output to the transmission unit 53.

Moreover, in the calculation unit 52, in addition to the user H getting into a bed (sleeping) and getting up from a bed (rising), for example, a calculation for predicting the user H getting up from a bed from a movement distance and/or a movement speed of a position of a center of gravity of the user H on the bed surface T of the bed main body 1A can be performed. In addition, it is possible to detect a movement of a body (for example, turning over in a bed or the like), a posture (for example, a supine position, a prone position, recumbent position, or the like), or the like of the user H by the calculation, and it is possible to predict occurrence of bedsores as described below.

The transmission unit 53 is a transmitter which is attached to the bed main body 1A, and transmits the results calculated by the calculation unit 52 to the reception unit 54 which is positioned separately from the calculation unit 52. Meanwhile, the reception unit 54 is a receiver which receives the signals transmitted from the transmission unit 53, and can remotely monitor the state (bed occupancy state) of the user H by receiving the signals from the transmission unit 53.

Moreover, in the reception unit 54 side, for example, results detected by the load cells 51 or results calculated by the calculation unit 52 may be displayed on a monitor (not shown) or may be printed by a printer.

In addition, for example, from the results calculated by the calculation unit 52, it is possible to notify a state of the user H to a guardian if necessary. A notification method is not particularly limited, and for example, an alarm may be generated from a speaker (not shown), or display may be performed on a monitor.

For example, the bed 1 provided with a load detection function having the above-described structure is suitably used in medical facilities (for example, hospitals, clinics, or the like), nursing facilities, care facilities, or the like.

In the present invention, by using the bed 1 having a load detection function, for example, it is possible to remotely monitor a state (bed occupancy state) of the user H such as having got into a bed (sleeping), having got up from a bed (rising), an in-bed position, a movement of a body (for example, turning over in a bed), or postures (for example, supine position, prone position, recumbent position, or the like). In addition, by using the bed 1 having a load detection function, it is possible to reduce a mental burden on the user H such as the user H being monitored by someone, or physical burdens and a mental burden on a guardian such as a case where the guardian must monitor the user H not only in the late evening or early morning but also all the times.

In addition, the use of the bed 1 having a load detection function is not limited to the above-described facilities. For example, the bed 1 having a load detection function may be also used in lodging facilities (for example, hotels, inns), ordinary households (for example, home care or the like). That is, a use of the bed 1 having a load detection function is not particularly limited.

In addition, for example, as an application example using a load detection function of the bed 1 having a load detection function to which the present invention is applied, there is a “bedsore prevention function”. Specifically, when a center does not move outside a certain circle in which a position of a center of gravity is positioned during a certain period of time (for example, two hours), or when a load of each load cell 51 does not change so as to remain a constant value (for example, 1 kg) or more, it is determined that there is a possibility that bedsores may occur in the user H, and a function of notifying this to a guardian can be added.

In addition, as another application example, there is an “illumination control function”. Specifically, by measuring the presence or absence of a weight of the user H on the bed surface T of the bed main body 1A, a position of a center of gravity of the user H, a movement amount of a center of gravity of the user H, a movement speed of a center of gravity of the user H, or the like, a function of turning on or turning off illumination when the user H gets into a bed or gets up from a bed can be added.

Moreover, as another application example, there is a “body weight management function”. Specifically, by measuring a body weight of the user H periodically (at a fixed time every day) on the bed surface T of the bed main body 1A, a function of performing a management of body weight of the user H can be added.

Moreover, as another application example, there is an “air-conditioning management function”. Specifically, by detecting a movement of a body (turning over in a bed, or the like) of the user H on the bed surface T of the bed main body 1A and measuring a sleeping depth of a user H, a function of managing air-conditioning according to a state of a user can be added.

In addition, as another application example, there is a “body weight monitor function in dialysis”. Specifically, by measuring a body weight of the user H on the bed surface T of the bed main body 1A, a function of detecting a start and end of dialysis can be added.

The present invention is not limited to the above-described functions, and various functions can be added using the load detection function of the bed 1 having a load detection function.

In addition, the present invention may be a bed having a load detection function in which the load detector 50 to which the present invention is applied is incorporated into the bed main body 1A in advance, or may be a bed having a load detection function in which the load detection function is added to an existing bed by separately incorporating the load detector 50 to which the present invention is applied into the bed main body 1A.

That is, in the bed having a load detection function to which the present invention is applied, it is possible to detect a state of the user H on the bed surface T of the bed main body 1A by measuring a change of a load applied to the bed main body 1A using the load detector 50 which is attached to the bed main body 1A in advance or is separately attached to the bed main body 1A.

Moreover, in the present invention, by attaching the load cell 51 of the load detector 50 to which the present invention is applied to the bed main body 1A, it is possible to add a load detection function to a bed with a simple structure while preventing an increase in the number of parts.

Specifically, in the load detector 50 to which the present invention is applied, since the load-receiving member 51A of the load cell 51 may be any member as long as the load-receiving member configures a load detection part which is exchangeable with a part (a bearing member on which a guide slit (bearing) 12 is formed) configuring the first hinge portion 10 of the first connection arm 9a included in an existing bed, it is possible to simply and easily incorporate the load cell 51 into the bed main body 1A.

Accordingly, it is possible to inexpensively add the load detection function to an existing bed. Moreover, even when faults or the like occur in the load cell 51, it is possible to easily replace the load cell 51. In addition, since a difference between a bed and an existing bed is small, the user H can use the bed without feeling uncomfortable.

In addition, the load-receiving member 51A and the base body 51B of the load cell 51 are not limited to the above-described examples, and various modifications may be applied within a scope which does not depart from the gist of the present invention.

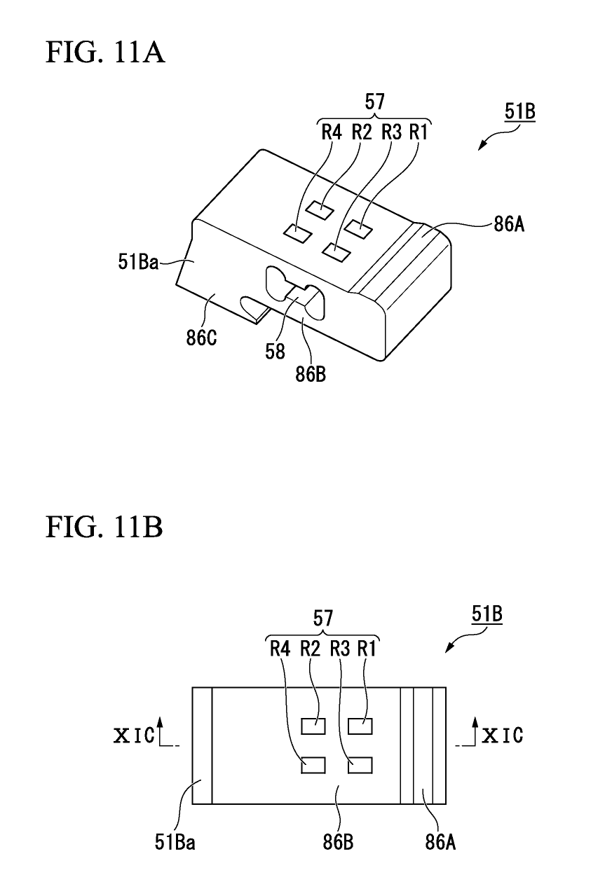

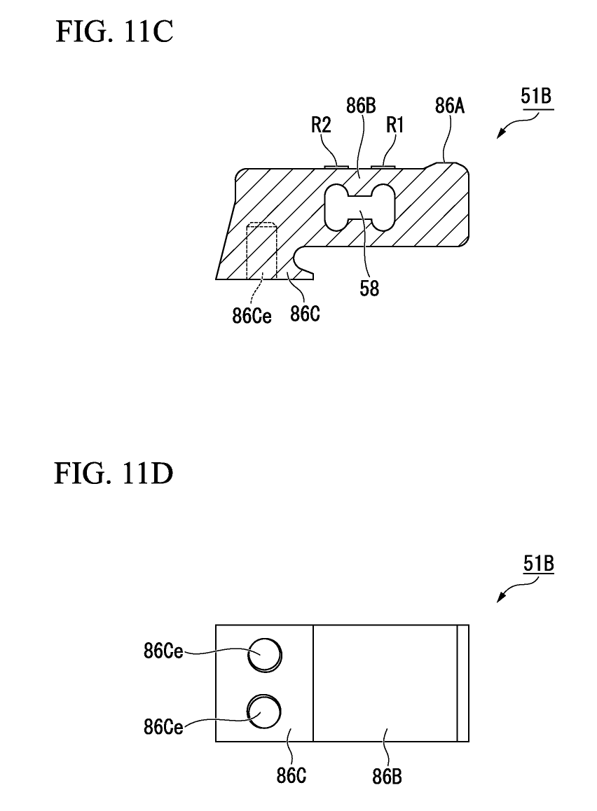

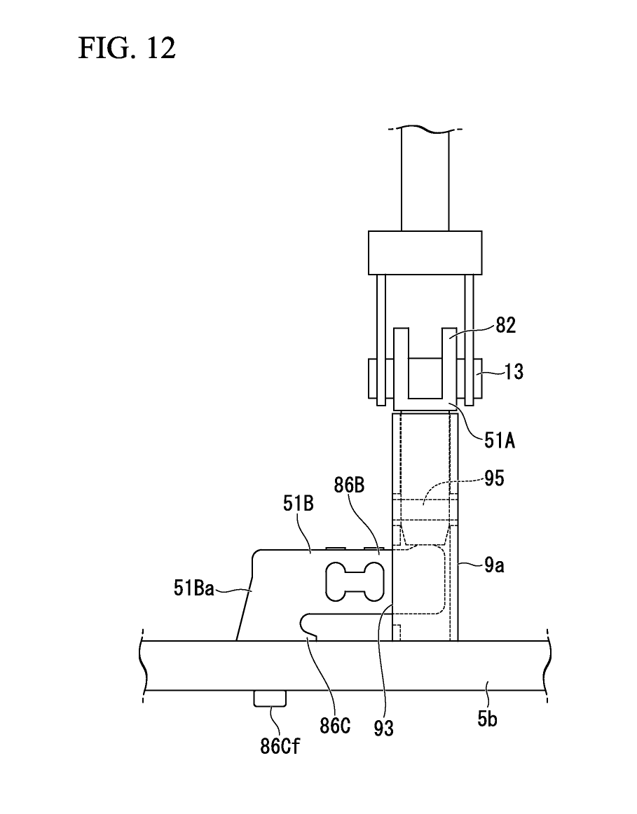

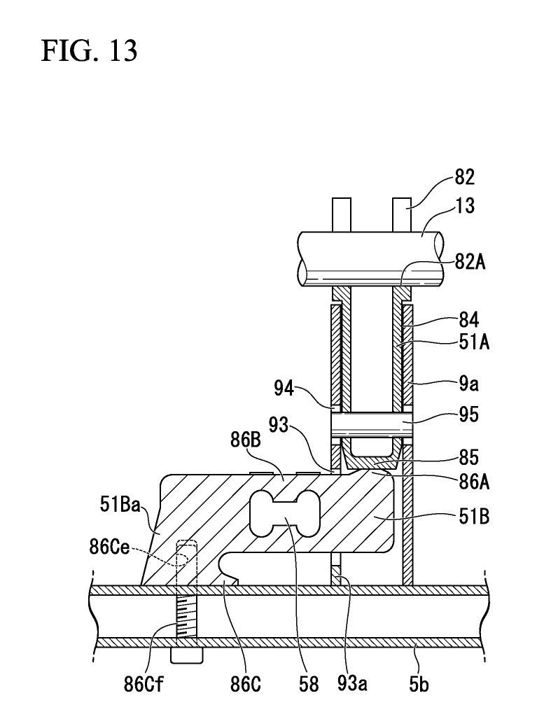

For example, a second example of the base body 51B of the load cell 51 is shown in FIGS. 11A to 11D, and an example in which the base body 51B of the second example and the first example (refer to FIGS. 6A to 6C) of the above-described load-receiving member 51A are combined with each other is shown in FIGS. 12 and 13.

In the base body 51B of the second example shown in FIGS. 11A to 11D, the action portion 86A and the operating portion (cantilever portion) 86B are substantially the same as those of the base body 51B of the first example shown in FIGS. 6A to 6C. However, the attachment portion 86C is different from that of the base body 51B of the first example.

That is, the attachment portion 86C is configured of a trapezoidal-shaped (pedestal-shaped) portion which is formed below the base end side portion (base portion 51Ba) of the operating portion (cantilever portion) 86B. Moreover, for example, two screw holes 86Ce are formed on the lower surface side of the pedestal-shaped attachment portion 86C from the lower side toward the upper side.

A state in which the base body 51B of the second example is combined with the load-receiving member 51A of the first example and the combined base body 51B and the load-receiving member 51A are incorporated into the square tube-shaped connection arm (cylindrical support body) 9a on the pipe 5b of the lower frame 5 in the bed main body 1A is shown in FIGS. 12 and 13.

In FIGS. 12 and 13, the base body 51B of the load cell 51 is placed on the upper surface of the pipe 5b so that the extension direction of the operating portion (cantilever portion) 86B from the base portion 51Ba is positioned along the length direction of the pipe 5b on the upper surface of the pipe 5b of the lower frame 5. In addition, a portion (a portion in which the action portion 86A is positioned) of the extended end side of the operating portion (cantilever portion) 86B in the base body 51B is inserted into the square tube-shaped connection arm 9a from the side opening portion 93 of the square tube-shaped connection arm 9a. Meanwhile, a portion of a base end side in the base body 51B (a portion of the base portion 51Ba side and a portion of the attachment portion 86C) is positioned outside the side opening portion 93 of the square tube-shaped connection arm 9a. In addition, a screw 86Cf is inserted into the screw hole 86Ce of the attachment portion 86C from the lower side or the inside of the pipe 5b and is screwed, and the base body 51B is fixed to the upper surface of the pipe 5b.

Even in the incorporated state shown in FIGS. 12 and 13, in the base body 51B of the load cell 51, only a portion including the action portion 86A is inserted into the square tube-shaped connection arm 9a, and the remaining portion protrudes outside the square tube-shaped connection arm 9a. In this way, effects generated by inserting only a portion of the base body 51B into the square tube-shaped connection arm 9a are similar to those of the above-described example.

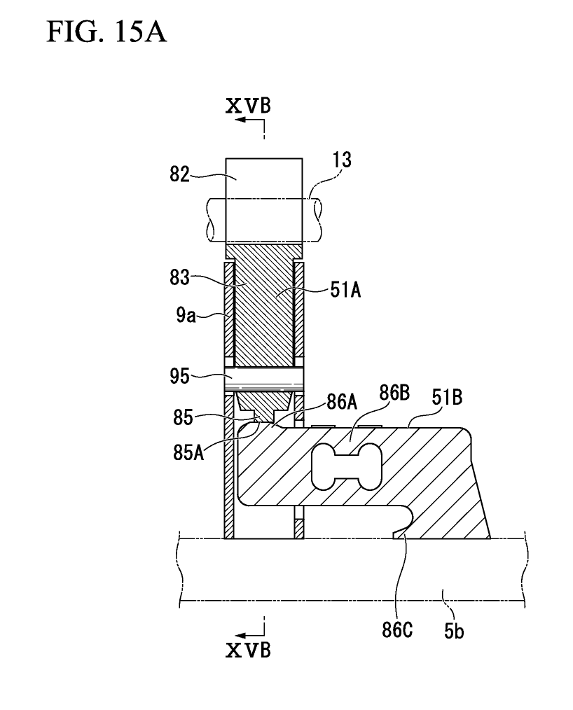

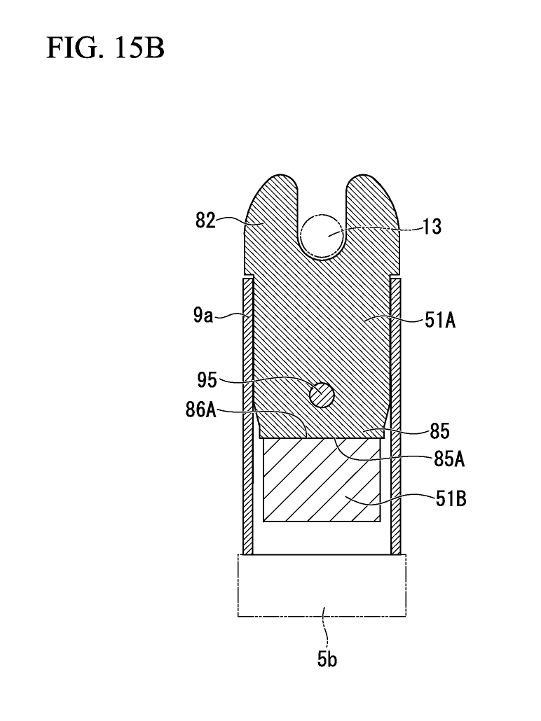

Meanwhile, a second example of the load-receiving member 51A of the load cell 51 is shown in FIGS. 14A to 14C, and an example in which the load-receiving member 51A of the second example and the second example (refer to FIGS. 11A to 11D) of the above-described base body 51B are combined with each other is shown in FIGS. 15A and 15B.

The main difference between the load-receiving member 51A of the second example shown in FIGS. 14A to 14C and the load-receiving member 51A of the first example shown in FIGS. 6A to 6C is the shape of the contact portion 85. That is, a width of the contact portion 85 of the second example is narrower than that of the contact portion 85 of the first example shown in FIGS. 6A to 6C, and a vertical section of the contact portion 85 of the second example is a protrusion portion formed in a rectangular shape, that is, a square bar-shaped portion which is laid in a vertical direction and has a narrow width. In this case, the contact area between the lower end surface (abutment surface) 85A of the contact portion 85 and the upper surface of the action portion 86A of the base body 51B is smaller than the contact area when the load-receiving member 51A of the first example is used. Accordingly, a friction resistance therebetween decreases, and as described above, a concern that the component of the force in the horizontal direction or the inclination direction in the unbalanced load state may be added to the base body 51B is decreased.

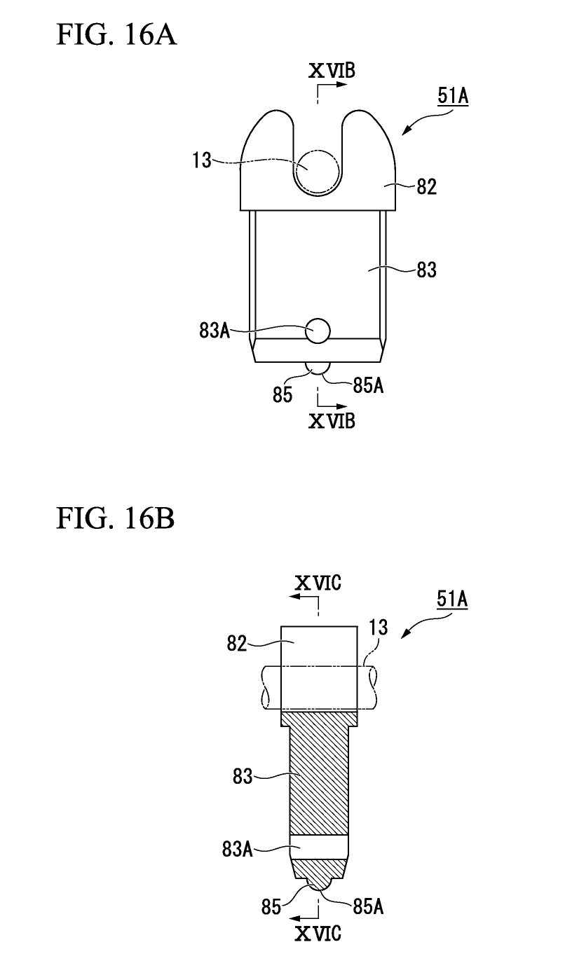

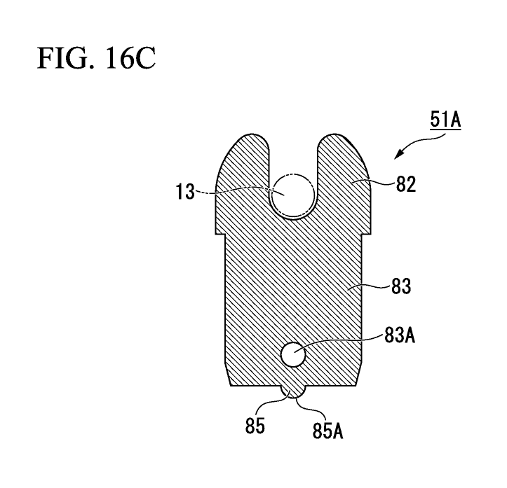

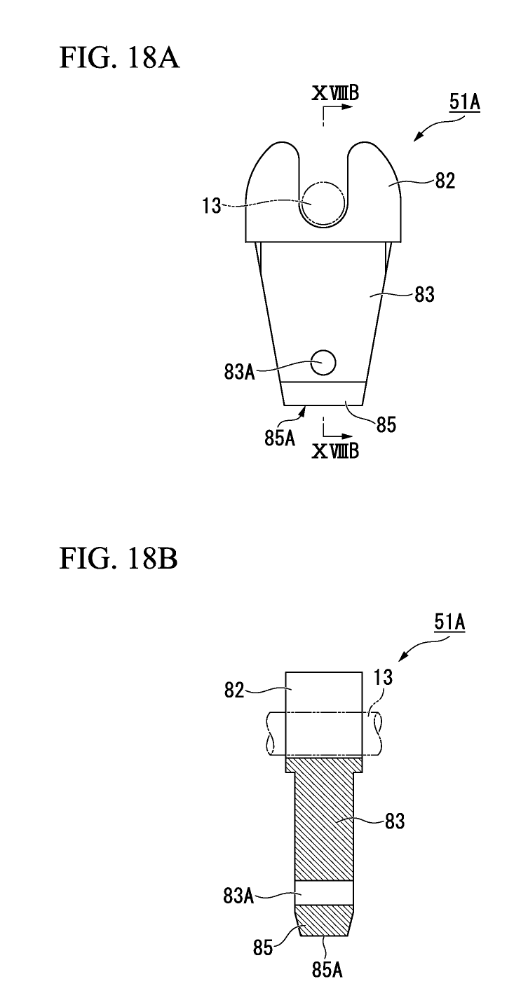

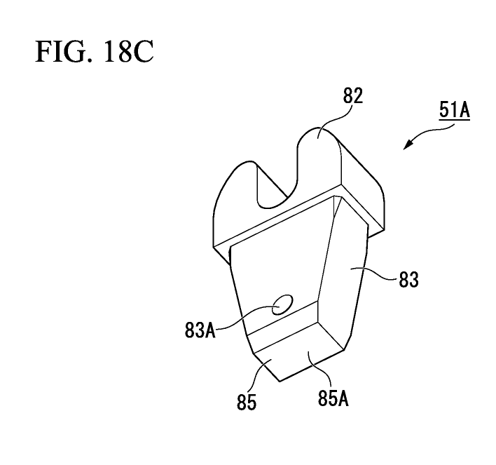

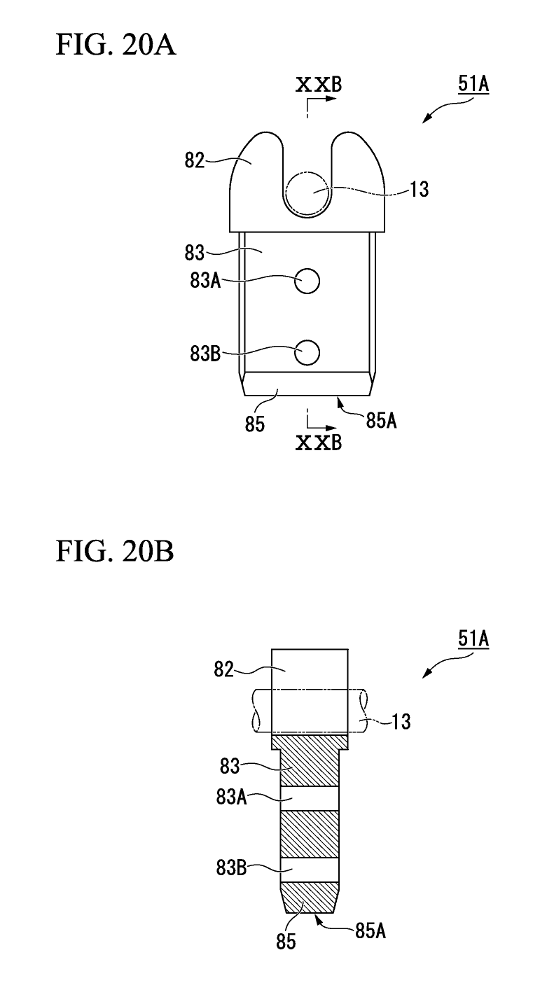

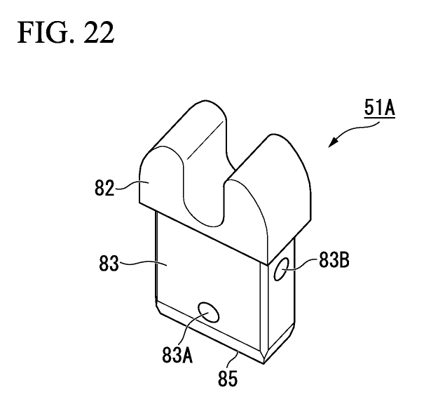

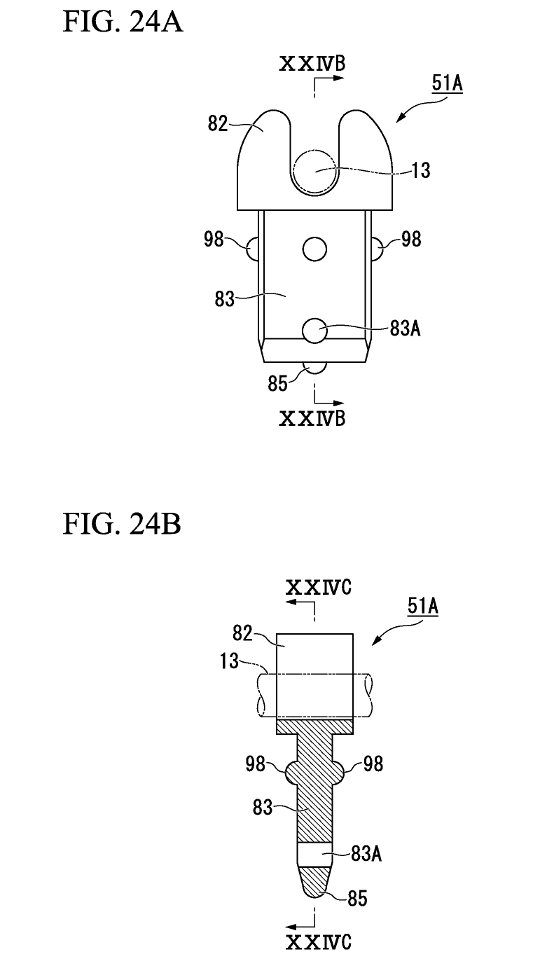

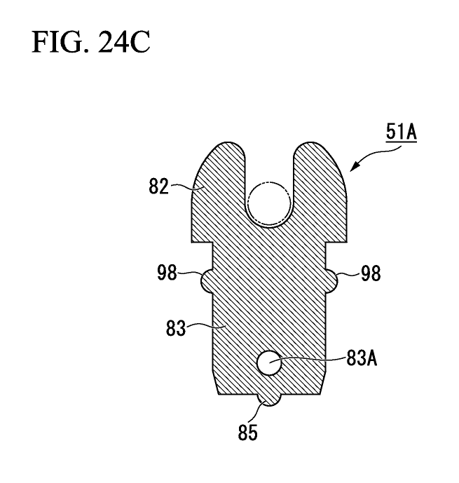

In addition, unlike the load-receiving member 51A of the first example shown in FIGS. 6A to 6C, in the load-receiving member 51A of the second example shown in FIGS. 14A to 14C, the space 51Ac, which is a portion surrounded by three side wall sites 51Aa, 51Ab, 51Ac and the bottom wall site 51Ad, is not formed, and the entire load-receiving member 51A of the second example is formed in a solid structure. However, similar to the load-receiving member 51A of the first example, the space 51A may be formed. This is similarly applied to the load-receiving member 51A of a third example shown in FIGS. 16A to 16C, the load-receiving member 51A of a fourth example shown in FIGS. 18A to 18C, the load-receiving member 51A of a fifth example shown in FIGS. 20A and 20B, the load-receiving member 51A of a sixth example shown in FIG. 22, and the load-receiving member 51A of a seventh example shown in FIGS. 24A to 24C described below.

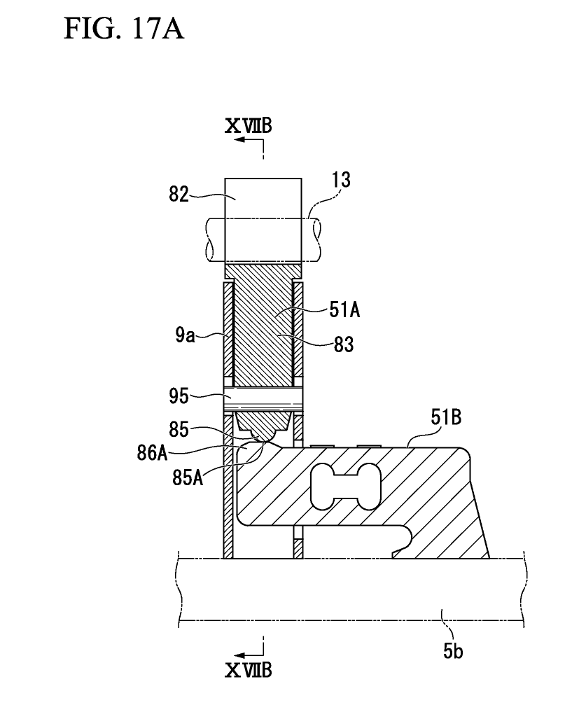

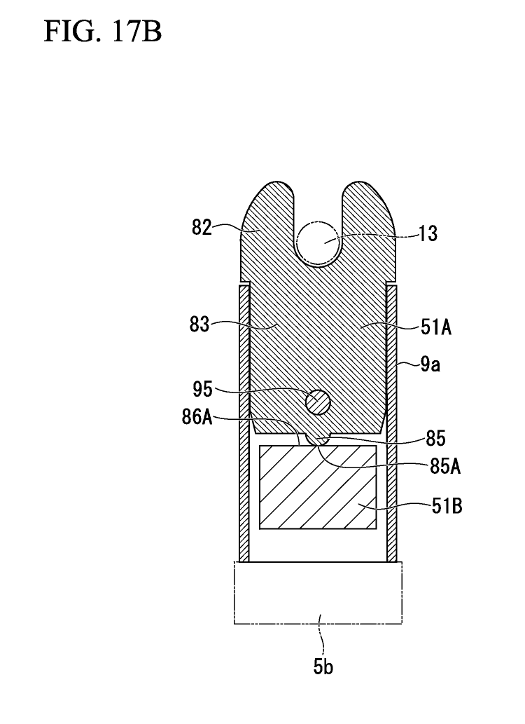

A third example of the load-receiving member 51A of the load cell 51 is shown in FIGS. 16A to 16C, and an example in which the load-receiving member 51A of the third example and the second example (refer to FIGS. 11A to 11D) of the above-described base body 51B are combined with each other is shown in FIGS. 17A and 17B.

In the load-receiving member 51A of the third example shown in FIGS. 16A to 16C, the contact portion 85 is formed in a portion which protrudes in a semispherical shape or a convex-curved surface shape (a portion of a spherical surface, a portion of a surface of an ellipsoid, or the like) downward in the vertical direction. In this case, the contact between the lower end surface (abutment surface) 85A of the contact portion 85 and the upper surface of the action portion 86A of the base body 51B substantially becomes a point contact, the contact area is significantly smaller than the contact area when the load-receiving member 51A of the first example is used, and the contact resistance therebetween is also decreased. Moreover, since a spherical surface (curved surface) and a plane surface come into contact with each other, even when the load-receiving member 51A is inclined by twisting or the like of the spindle (pin) 13, the inclination of the load-receiving member 51A does not influence the base body 51B side, and only the component in the vertically downward direction is applied to the base body 51B. Accordingly, a concern that the force in the horizontal direction or the inclination direction in the unbalanced load state may be applied to the base body 51B is decreased, and it is possible to detect the load in the vertically downward direction with higher accuracy.

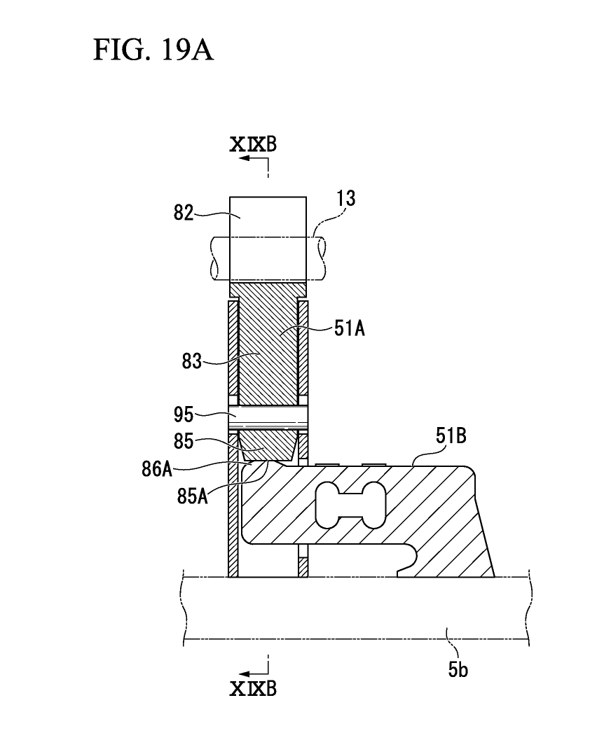

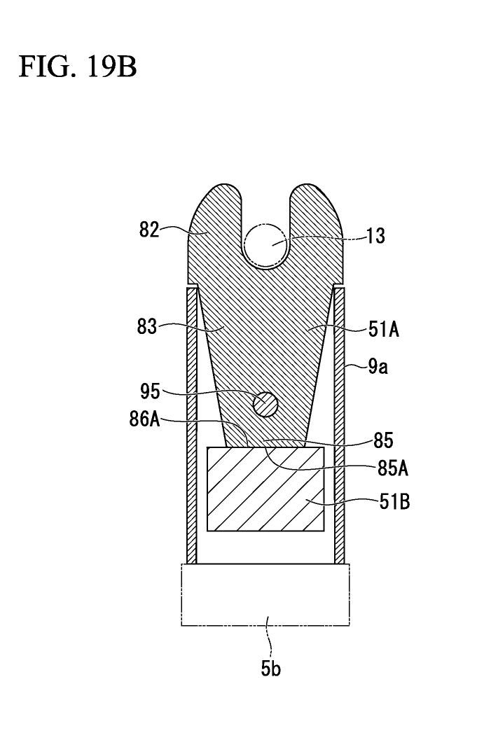

In addition, a fourth example of the load-receiving member 51A of the load cell 51 is shown in FIGS. 18A to 18C, and an example in which the load-receiving member 51A of the fourth example and the second example (refer to FIGS. 11A to 11D) of the above-described base body 51B are combined with each other is shown in FIGS. 19A and 19B.

In the load-receiving member 51A of the fourth example shown in FIGS. 18A to 18C, an outer surface of the load-receiving member 51A is formed in a taper shape so that the dimension of the horizontal section of the insertion portion 83 decreases downward, and the contact portion 85 in which the dimension of the horizontal section also decreases downward and which has a downward trapezoidal shape is formed on the lower end of the insertion portion 83. Even in the load-receiving member 51A, an area of the lower end surface (abutment surface) 85A of the contact portion 85 is small, and accordingly, as described above, a concern that the component of the force in the horizontal direction or the inclination direction in the unbalanced load state may be applied to the base body 51B is decreased.

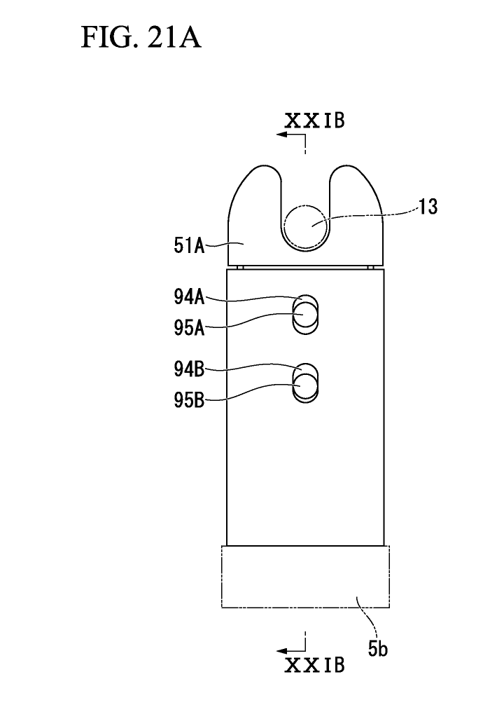

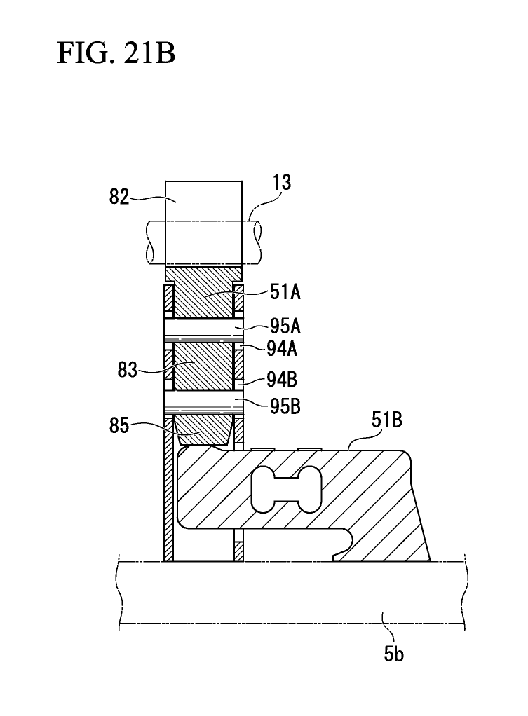

Moreover, a fifth example of the load-receiving member 51A of the load cell 51 is shown in FIGS. 20A and 20B, and an example in which the load-receiving member 51A of the fifth example and the second example (refer to FIGS. 11A to 11D) of the above-described base body 51B are combined with each other is shown in FIGS. 21A and 21B.

In the load-receiving member 51A of the fourth example shown in FIGS. 20A and 20B, shaft holes 83A and 83B are formed at a plurality of locations on the insertion portion 83, for example, at two upper and lower locations. In this case, as shown in FIGS. 21A and 21B, long holes 94A and 94b are formed on the side wall of the square tube-shaped connection arm 9a at two upper and lower locations, and both ends of auxiliary pins 95A and 95B inserted into the shaft holes 83A and 83B of the load-receiving member 83 are inserted into the long holes 94A and 94B of the square tube-shaped connection arm 9a. Accordingly, in this example, a posture of the load-receiving member 51A is held by the two auxiliary pins 95A and 95B and the two long holes 94A and 94B corresponding to the two auxiliary pins 95A and 95B.

In this way, when a plurality of auxiliary pins and a plurality of long holes corresponding to the plurality of auxiliary pins are provided, it is possible to stabilize a posture of the load-receiving member 51A even when a load is greatly changed or a biased load is applied.

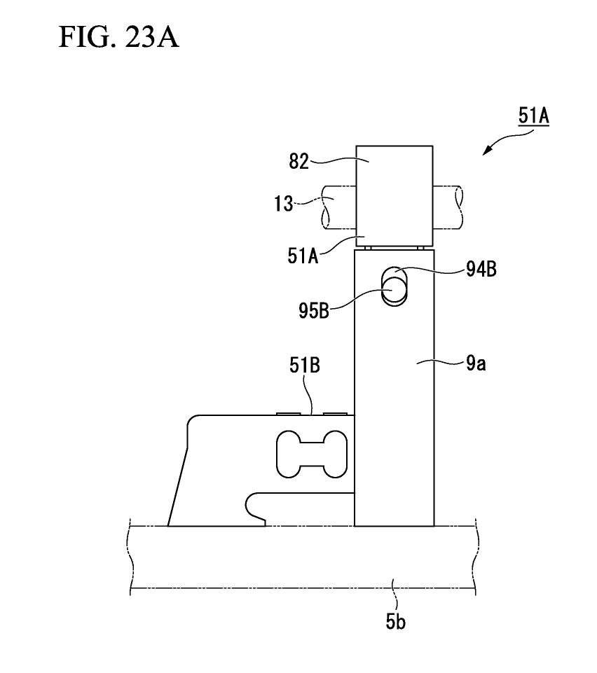

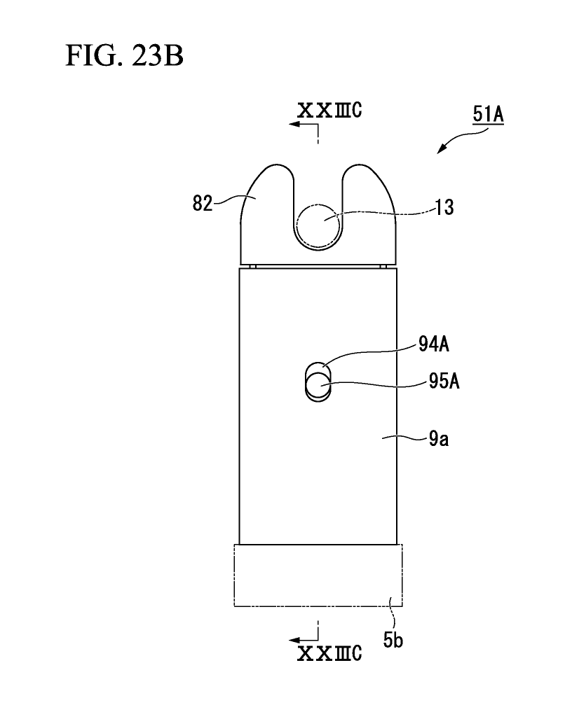

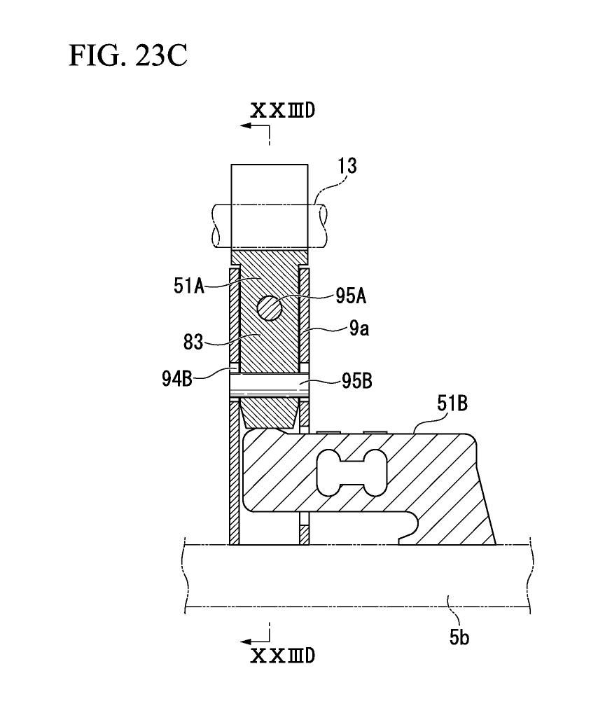

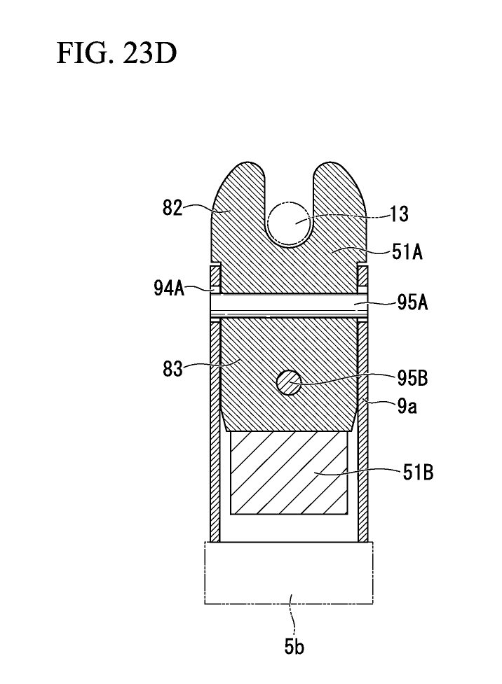

In addition, a sixth example of the load-receiving member 51A of the load cell 51 is shown in FIG. 22, and an example in which the load-receiving member 51A of the sixth example and the second example (refer to FIGS. 11A to 11D) of the above-described base body 51B are combined with each other is shown in FIGS. 23A to 23D.

In the load-receiving member 51A of the sixth example shown in FIG. 22, shaft holes 83A and 83B are formed along directions different from each other by 90° at two upper and lower locations of the insertion portion 83. That is, one shaft hole 83A is formed between two side surfaces of a narrower width side of the insertion portion 83 along a direction orthogonal to the axial direction (that is, the axial direction of the spindle 13) of the bearing surface 82A in the bearing portion 82, and the other shaft hole 83B is formed between two side surfaces of a wider width side of the insertion portion 83 along the axial direction (that is, the axial direction of the spindle 13) of the bearing surface 82A in the bearing portion 82.

In this case, as shown in FIGS. 23A to 23D, long holes 94A and 94B are formed on two surface of the narrower width side of the side walls of the square tube-shaped connection arm 9a and on two surfaces of the wider width side. In addition, two ends of one auxiliary pin 95A inserted into the one shaft hole 83A of the load-receiving member 83 are inserted into the long hole 94A of the narrower width side of the square tube-shaped connection arm 9a, and two ends of the other auxiliary pin 95B inserted into the other shaft hole 83B of the load-receiving member 83 are inserted into the long hole 94A of the wider width side of the square tube-shaped connection arm 9a. Accordingly, in this example, a posture of the load-receiving member 51A is held on sides in directions different from each other by 90°. Therefore, even when the load-receiving member 51A is inclined in any direction due to a biased load as described above, the posture of the load-receiving member 51A is stabilized.

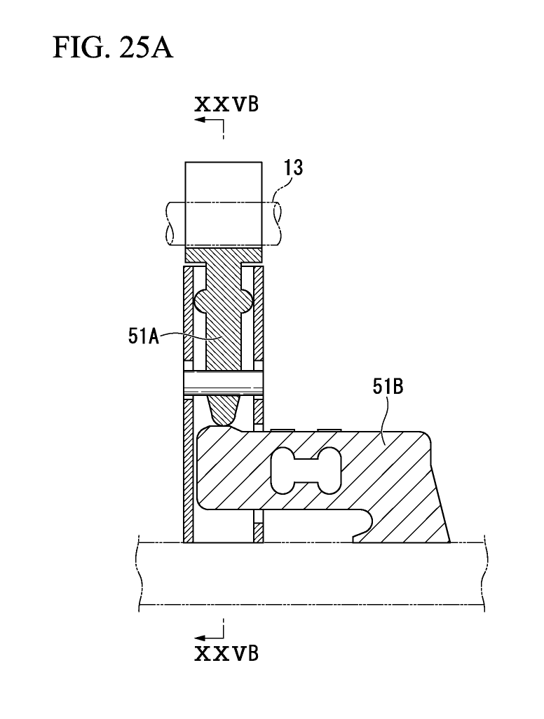

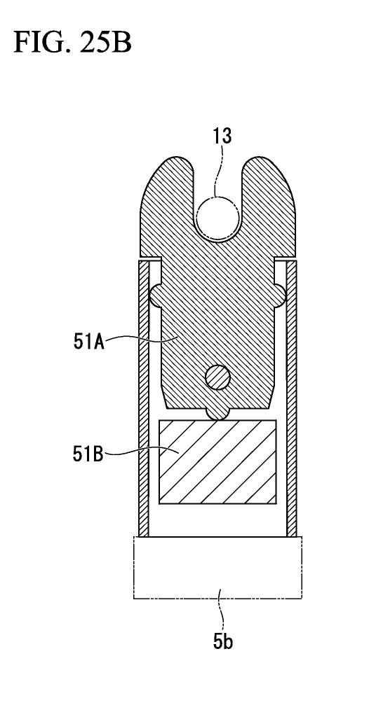

Moreover, a seventh example of the load-receiving member 51A of the load cell 51 is shown in FIGS. 24A to 24C, and an example in which the load-receiving member 51A of the seventh example and the second example (refer to FIGS. 11A to 11D) of the above-described base body 51B are combined with each other is shown in FIGS. 25A and 25B.

In the load-receiving member 51A of the seventh example shown in FIGS. 24A and 24B, for example, protrusion portions 98 protruding in the horizontal direction are formed on four side surfaces of the upper portion of the insertion portion 83. Preferably, friction resistances between tips of the protrusion portions 98 and the inner surface of the square tube-shaped connection arm (cylindrical support member) 9a are small. Accordingly, in the present example, each of the protrusion portions 98 is formed in a semispherical shape or a convex-curved shape, and a contact state between the protrusion portion 98 and the inner surface of the connection arm 9a substantially becomes a point contact.

In this way, if the protrusion portions 98 are formed on four side surfaces of the insertion portion 83 of the load-receiving member 51A, it is possible to prevent a position of the load-receiving member 51A from deviating inside the square tube-shaped connection arm (cylindrical support member) 9a. That is, when the position of the load-receiving member 51A deviates inside the square tube-shaped connection arm 9a and the side surface of the insertion portion 83 of the load-receiving member 51A comes into surface-contact with the inner wall of the square tube-shaped connection arm 9a, the load in the vertically downward direction of the load-receiving member 51A is applied to the square tube-shaped connection arm 9a due to the friction resistance therebetween. As a result, the load in the vertically downward direction transmitted to the base body 51B is decreased, and there is a concern that detection accuracy of a load may deteriorate. However, as described above, the protrusion portions 98 are formed, and the friction resistances between the tips of the protrusion portions 98 and the inner wall of the square tube-shaped connection arm 9a are decreased. Accordingly, it is possible to prevent the position of the load-receiving member 51A from deviating, and it is possible to prevent the load in the vertically downward direction transmitted to the base body 51B from being decreased.

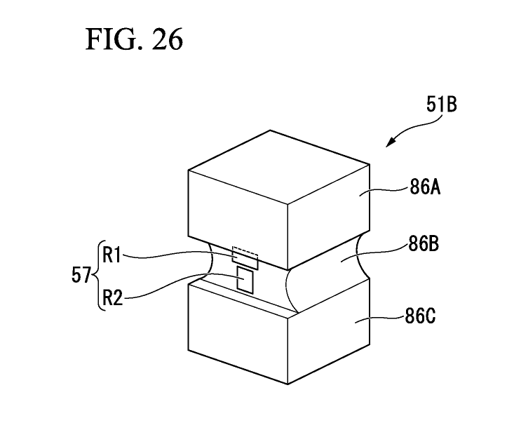

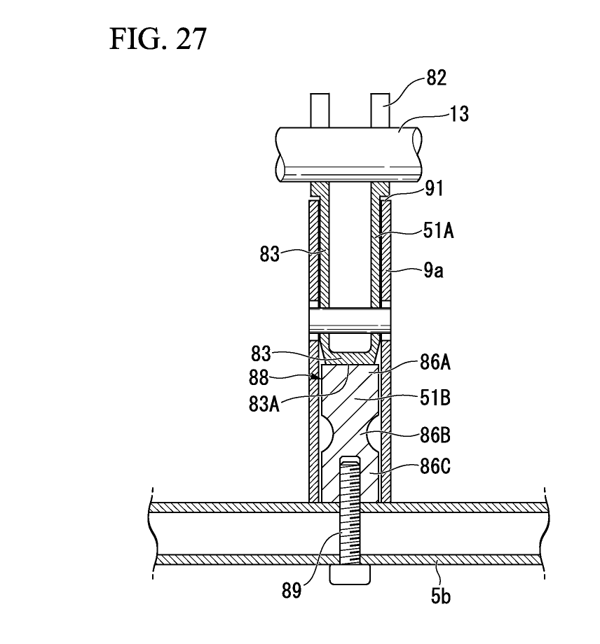

In each of the above-described examples, a configuration of a cantilever type strain element is adopted by the base body 51B of the load cell 51. However, in some cases, a so-called compression type strain element may be adopted. An example (third example) of the base body 51B which is configured as a compression type is shown in FIG. 26, and a state is shown in FIG. 27 in which the base body 51B of the third example is combined with the load-receiving member 51A (refer to FIGS. 6A to 6C) of the first example and the combined base body 51B and load-receiving member 51A are incorporated into the square tube-shaped connection arm (cylindrical support body) 9a on the pipe 5b of the lower frame 5 in the bed main body 1A.

The base body 51B shown in FIG. 26 is compressed and strained in the vertical direction, and strain of the side surface portions is detected by the strain sensor 57. Specifically, in the base body 51B, for example, an upper portion which is formed in a rectangular parallelepiped shape comes into contact with the contact portion 85 of the load-receiving member 51A, and corresponds to the action portion 86A on which a load from the load-receiving member 51A acts. Similarly, for example, a lower portion of the base body 51B which is formed in rectangular parallelepiped shape corresponds to the attachment portion 86C which is fixed to a configuration member (in the case of the present example, the pipe 5b of the lower frame 5 or the square tube-shaped connection arm 9a on the pipe 5b) of the installation surface side in the bed main body 1A. Moreover, an intermediate portion between the upper rectangular parallelepiped-shaped action portion 86A and the lower rectangular parallelepiped-shaped attachment portion 86C becomes the operating portion 86B which is recessed in a drum shape from the side surface sides, and a plurality of strain gauges (strain sensitive resistors) R1 and R2 configuring the strain sensor 57 adhere to the side surface (the surface which is recessed in a drum shape) of the operating portion 86B. In addition, in this case, as a material of the base body 51B, for example, a metal such as aluminum alloy, iron, steel, or titanium alloy, or an elastic compression-deformable material such as a hard resin such as an engineering plastic or hard rubber are used.

When a load is applied to the upper surface of the upper rectangular parallelepiped-shaped action portion 86A in a vertically downward direction, the surface of the operating portion 86B which is recessed in a drum shape is compressed in a vertical direction and extends in a horizontal direction. Accordingly, by appropriately determining directions of the plurality of strain gauges (strain sensitive resistors) adhering to the surface of the operating portion 86B, strain of the operating portion 86B is detected using the above-described Wheatstone bridge circuit or the like. As a result, it is possible to detect a change of the load applied to the action portion 86A. In addition, in FIG. 26, only two strain gauges (R1 and R2) are shown. However, if some strain gauges adhere to the surfaces of other sides (or other locations on the same side) of the operating portion 86B or some dummy resistances are used, the above-described Wheatstone bridge circuit can be configured.

For example, when the above-described compression type base body 51B is combined with the load-receiving member 51A (refer to FIGS. 6A to 6C) of the first example and the combined based body 51B and load-receiving member 51A are incorporated into the square tube-shaped connection arm 9a on the pipe 5b of the lower frame 5 in the bed main body 1A, as shown in FIG. 27, the entire base body 51B is inserted into the square tube-shaped connection arm 9a. In addition, a gap 88 is maintained between the side wall surface of the base body 51B and the inner wall surface of the square tube-shaped connection arm 9a. In addition, the attachment portion 86C of the compression type base body 51B may be fixed to the pipe 5b or the square tube-shaped connection arm 9a. Here, a method for fixing the attachment portion 86C is not particularly limited. However, in the case of FIG. 27, the attachment portion 86C is fixed to the pipe 5b or the square tube-shaped connection arm 9a from the inside or the lower side of the pipe 5b by a screw 89.

In addition, similarly to each of the above-described examples, the insertion portion 83 of the load-receiving member 51A is inserted from the upper opening end 91 of the square tube-shaped connection arm 9a, and the lower end surface (abutment surface) 85A of the contact portion 85 of the load-receiving member 51A comes into contact with the upper surface of the action portion 86A of the base body 51B.

In this way, even in the case where the compression type base body 51 is used for the base body 51A of the load cell 51, similarly to those described above, a load applied from the bed surface-forming portion 100 of the bed main body 1A is applied to the load-receiving member 51A of the load cell 51 via the spindle 13, the load of the component in the vertically downward direction of the load is applied to the base body 51B, the operating portion 86B is compressed and deformed, and strain on the surface of the operating portion 86B is detected by the strain sensor 57.

In addition, in each example of the above-described load cells 51, as the strain sensor for detecting the magnitude of the strain, the configuration which uses the strain gauge (strain sensitive resistor) 57 is adopted. However, the present invention is not limited to the strain sensitive resistor, and for example, a conductive elastomeric sensor, an optical strain sensor, an electrostrictive device sensor, a piezoelectric device sensor, a magnetostrictive device sensor, or the like may be used.

Moreover, in the bed main body 1A, a mat or the like may be laid on the bed plate 2 in advance. In addition, the bed plate 2 may have a structure in which the bed plate 2 is divided in the length direction (longitudinal direction of the bed main body 1A), and may have a reclining function in which an upper half body side or a portion of a foot side of the user H gets up. In addition, the structures of the upper frame 3 and the lower frame 5 are not limited to the above-described frame structures, and may adopt various frame structures.

In addition, the load detector 50 is not limited to the configuration in which the portion between the load cell 51 and the calculation unit 52 is electrically connected by the wire 55a and the portion between the calculation unit 52 and the transmission unit 53 is electrically connected by the wire 55b. That is, for example, the portions may be electrically connected using a wireless system. Meanwhile, a communication method between the transmission unit 53 and the reception unit 54 is not limited to the above-described a wireless communication network, and a wired communication network may be used. Moreover, in the load detector 50, the calculation unit 52 and the transmission unit 53 may be integrally formed.

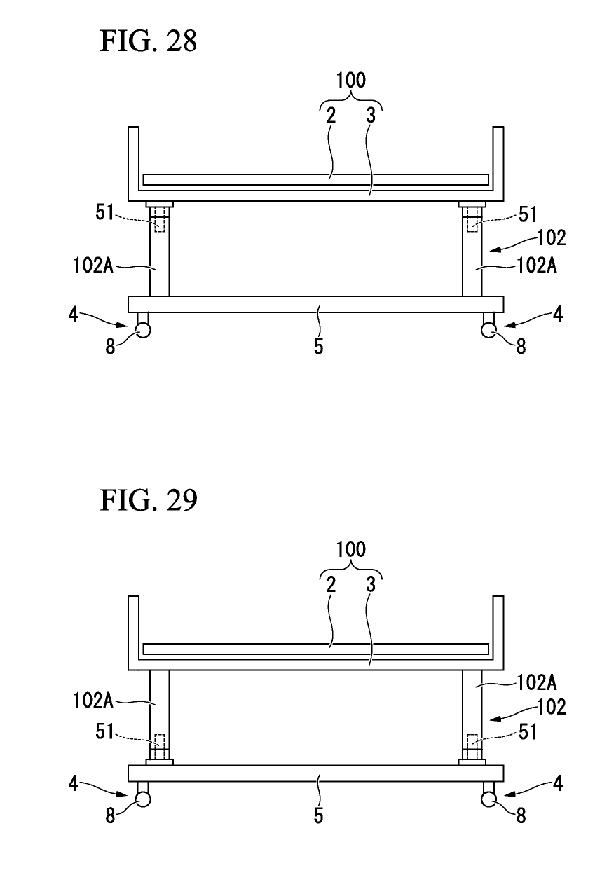

In addition, in the above-described example shown in FIGS. 1, 2A, and 2B, the lifting-lowering link mechanism 6 is provided in the connection-support portion 102 between the upper frame 3 and the lower frame 5. However, the present invention may be also applied to a case where the lifting-lowering link mechanism 6 is not provided in the connection-support portion 102. An example of these is shown in FIG. 28.

In the example shown in FIG. 28, for example, a portion between the upper frame 3 and the lower frame 5 is connected by a plurality of (in general, four) hollow pipe-shaped vertical columns 102A serving as the connection-support portion 102, and the load cell 51 is interposed between an upper end of each column 102A and the upper frame 3. In this case, each column 102A corresponds to the configuration member of the installation surface side described in the first aspect.

In addition, another example in which the lifting-lowering link mechanism 6 is not provided in the connection-support portion 102 is shown in FIG. 29.

Similarly to the example shown in FIG. 28, in the example shown in FIG. 29, the portion between the upper frame 3 and the lower frame 5 is connected by the plurality of (in general, four) hollow pipe-shaped vertical columns 102A serving as the connection-support portion 102, for example. However, in this case, the load cell 51 is interposed between a lower end of each column 102A and the lower frame 5. In this case, the lower frame 5 corresponds to the configuration member of the installation surface side described in the first aspect.

Moreover, the example shown in FIG. 28 and the example shown in FIG. 29 describe the case where the lifting-lowering link mechanism is not provided in the connection-support portion 102 between the upper frame 3 and the lower frame 5. However, even in the case where the lifting-lowering link mechanism is provided in the connection-support portion 102, according to the example shown in FIG. 28, the load cell 51 may be interposed between the upper frame 3 and the connection-support portion 102 (for example, between the upper frame 3 and the lifting-lowering link mechanism). In addition, similarly, even in the case where the lifting-lowering link mechanism is provided in the connection-support portion 102, according to the example shown in FIG. 29, the load cell 51 may be interposed between the connection-support portion 102 and the lower frame 5 (for example, between the lifting-lowering link mechanism and the lower frame 5).

Moreover, as the example shown in FIG. 28 or the example shown in FIG. 29, in the case where the lifting-lowering link mechanism is not provided in the connection-support portion 102 between the upper frame 3 and the lower frame 5, the load cell 51 may be interposed at an intermediate portion of each column 102A configuring the connection-support portion 102.

Meanwhile, in the present invention, the load cells 51 for detecting a load of the bed main body may be disposed in four leg portions 4 of the bed main body 1A. That is, in this kind of bed main body 1A, in general, the caster mechanism 8 for easily moving the bed main body 1A is provided in each leg portion 4. However, the load cell 51 may be interposed in the portion in which the cater mechanism 8 is accommodated or the inner portion of the caster mechanism 8.

In addition, when the lifting-lowering link mechanism 6 is not provided (for example, refer to the example shown in FIG. 28), the lower frame 5 is also omitted. In this case, the caster mechanism 8 serving as the leg portion 4 may be directly provided on the lower end of each column 102A. Even in the bed main body having the above-described configuration, according to the example shown in FIG. 28, the load cell 51 may be interposed between the upper frame 3 and each column 102A, or the load cell 51 may be interposed in the leg portion 4 (for example, caster mechanism 8).

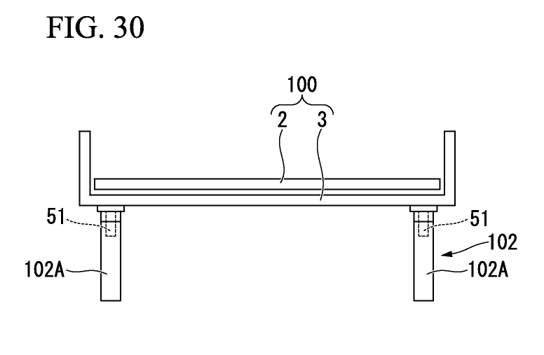

Moreover, the present invention may be applied to a bed main body which does not include the lifting-lowering link mechanism and the caster mechanism. An example of this case is shown in FIG. 30. In this example, the load cell 51 is interposed between the upper frame 3 and each column 102A corresponding to the leg portion. In this case, each column 102A corresponds to the configuration member of the installation surface side described in the first aspect.

As described above, in the bed 1 having a load detection function to which the present invention is applied, the load cell 51 is incorporated into the site which is positioned at any location on the load transmission path extending from the bed surface-forming portion (configured by the bed plate 2 and the upper frame 3 in the above-described each embodiment) 100 to the leg portion 4 via the connection-support portion (regardless of the presence or absence of the lifting-lowering link mechanism 6 or the lower frame 5) 102, and at which a load from the bed surface-forming portion 100 side is received and the load is transmitted to the installation surface B side. Accordingly, the load cell 51 may be interposed at any of the portion between the bed surface-forming portion 100 and the connection support portion 102, the intermediate portion of the connection-support portion 102, a portion between the connection-support portion 102 and the leg portion 4, or the portion of the leg portion 4.

Moreover, in each of the above-described examples, the bed surface-forming portion 100 forming the bed surface T in the bed main body 1A is configured of the bed plate 2, and the upper frame 3 supporting the bed plate 2. However, in some cases, the bed surface-forming portion 100 may not include the upper frame 3, that is, may include only the bed plate 2. Even in this case, the present invention may be applied. For example, the load cell 51 may be interposed between the bed plate 2 and the connection-support portion (for example, column 102A) for supporting the bed plate 2.

In addition, as described above, in the case where the bed surface-forming portion 100 does not include the upper frame 3, that is, includes only the bed plate 2, the bed main body may be configured so that the lifting-lowering link mechanism 6 is provided in the connection-support portion 102 for supporting the bed plate 2 and the bed plate 2 is directly lifted, and even in this case, the present invention may be applied.

In addition, the bed main body may be configured so that the upper frame 3 functions as only a fence even when the bed surface-forming portion 100 includes the upper frame 3 and the lifting-lowering link mechanism 6 directly lifts and lowers the bed plate 2. In this case, since the upper frame 3 does not substantially support a load, the upper frame 3 is deviated from the load transmission path extending from the bed surface-forming portion 100 to the leg portion 4 via the connection-support portion 102. In addition, in this case, the load cell 51 may be interposed at any location on the load transmission path extending from the bed plate 2 to the leg portion 4 via the connection-support portion 102.

In addition, in the above descriptions, the link mechanism is applied to the mechanism for lifting and lowering the bed surface-forming portion 100. However, in some cases, a lifting-lowering mechanism which does not use a link mechanism, for example, a lifting-lowering mechanism such as a rotational screw type (screw type) which is manually or electrically driven or a jack type may be used, and the present invention may be also applied to a bed main body having a lifting-lowering mechanism other than the above-described link mechanisms.

A bed having a load detection function according to the present invention can be used in medical facilities (for example, hospitals, clinics, or the like), nursing facilities, care facilities, lodging facilities (for example, hotels, and inns), ordinary households (for example, home care or the like), or the like. In this case, by detecting a load applied to a bed, for example, it is possible to detect states (bed occupancy states) of a bed user such as having got into a bed (sleeping), having got up from a bed (rising), an in-bed position, a movement of a body (for example, turning over in a bed), or postures (for example, supine position, prone position, recumbent position, or the like). In addition, the bed-load detector according to the present invention can be incorporated into not only a new bed but also an existing bed, and even in the case where the bed-load detector is incorporated into an existing bed, the above-described functions can be utilized.

### EXPLANATION OF REFERENCES

1: bed having a load detection function
1A: bed main body
2: bed plate
3: upper frame
4: leg portion
5: lower frame
6: lifting-lowering link mechanism
8: caster mechanism
9a: first connection arm
9b: second connection arm (cylindrical support member)
9c: third connection arm
9d: fourth connection arm
10a: first hinge portion
10b: second hinge portion
10c: third hinge portion
10d: fourth hinge portion
13: pin (spindle)
50: load detector
51: load cell
51A: load-receiving member
51B: base body
57: strain sensor
82: bearing portion
83: insertion portion
84: gap
85: contact portion
86A: action portion
86B: operating portion
86C: attachment portion
73: operating portion
75: load-receiving portion
100: bed surface-forming portion
102: connection-support portion
B: installation surface
T: bed surface
H: user
R₁, R₂, R₃, and R₄: strain gauge (resistor)

1. A bed having a load detection function which detects a change of a load applied to a bed main body by a load detector attached to the bed main body and detects a state of a user on a bed surface of the bed main body,
wherein the bed main body is configured to include a bed surface-forming portion which forms the bed surface, a leg portion which comes into contact with an installation surface on which the bed main body is to be installed, and a connection-support portion which connects the bed surface-forming portion and the leg portion such that the bed surface-forming portion is positioned above the installation surface, and transmits a load from the bed surface-forming portion to the leg portion,
wherein the load detector includes a load cell which measures strain generated when a load is applied to the bed main body,
wherein the load cell is provided at a site which is positioned at any location on a load transmission path extending from the bed surface-forming portion to the installation surface via the connection-support portion, and at which a load from the bed surface-forming portion side is received and the load is transmitted to a configuration member of the installation surface side,
wherein the load cell includes a load-receiving member which receives a load from the bed surface-forming portion side, and a base body which is separated from the load-receiving member and to which the load from the load-receiving member is applied, and
wherein the base body is configured to include an action portion with which the load-receiving member comes into contact and on which a load from the load-receiving member acts, an operating portion which is strain-deformed by the load applied to the action portion, a strain sensor which is attached to the operating portion, and an attachment portion which is continuous with the operating portion and is fixed to the configuration member of the installation surface side in the bed main body.

2. The bed having a load detection function according to claim 1,
wherein the operating portion in the base body of the load cell is configured of a flexibly deformable cantilever portion in which one end is continuous with the action portion and the other end is continuous with the attachment portion.

3. The bed having a load detection function according to claim 1,
wherein the operating portion in the base body of the load cell is configured of a compression-deformable member in which one end is continuous with the action portion and the other end is continuous with the attachment portion.

4. The bed having a load detection function according to claim 1,
wherein a spindle having a substantially horizontal axial line is placed in the load transmission path of the bed main body, and a bearing portion which rotatably supports the spindle is formed on the load-receiving member of the base body.

5. The bed having a load detection function according to claim 1,
wherein the connection-support portion of the bed main body includes a lower frame which is substantially parallel with the installation surface, the leg portion is provided on the lower frame, the lower frame is a configuration member of the installation surface side, and the cylindrical support body is fixed to the lower frame.

6. The bed having a load detection function according to claim 1,
wherein a hollow cylindrical support body is perpendicularly fixed to the configuration member of the installation surface side in the bed main body such that one end opening portion of the hollow cylindrical support body faces upward, a lower portion of the load-receiving member is inserted into an opening portion of the cylindrical support body from above, and at least a portion of the base body is inserted into a lower portion of the cylindrical support body such that the action portion of the base body is positioned inside the cylindrical support body and the action portion and the operating portion do not come into contact with the cylindrical support body.

7. The bed having a load detection function according to claim 2,
wherein a hollow cylindrical support body is perpendicularly fixed to the configuration member of the installation surface side in the bed main body such that one end opening portion of the hollow cylindrical support body faces upward, a lower portion of the load-receiving member is inserted into an opening portion of the cylindrical support body from above, an opening window portion is formed on a side surface side of a lower portion of the cylindrical support body, a portion of the base body is inserted into the cylindrical support body from the opening window portion such that at least the action portion is positioned inside the cylindrical support body and the action portion and the operating portion do not come into contact with the cylindrical support body, and the remaining portion of the base body is positioned outside the cylindrical support body.

8. The bed having a load detection function according to claim 7,
wherein at least a portion of the attachment of the base body is inserted into the cylindrical support body, and the attachment portion is fixed to an inner wall surface of the cylindrical support body.

9. (canceled)

10. The bed having a load detection function according to claim 7,
wherein the connection-support portion of the bed main body includes a lower frame which is substantially parallel with the installation surface, the leg portion is provided on the lower frame, the lower frame is configured by combining at least four pipes, one pipe or two or more pipes are configuration members of the installation surface side, the cylindrical support body is fixed to one pipe or each of two or more pipes, and the base body is disposed such that a length direction of the cantilever portion is along a length direction of the pipe.

11. The bed having a load detection function according to claim 1,
wherein the load cell is placed at an intermediate portion of the connection-support portion.

12. The bed having a load detection function according to claim 11,
wherein the connection-support portion includes a lifting-lowering link mechanism which lifts and lowers the bed surface-forming portion, and
wherein the load cell is incorporated into the lifting-lowering link mechanism.

13. The bed having a load detection function according to claim 11,
wherein the connection-support portion includes a lower frame which is supported above the installation surface via the leg portion, in addition to the lifting-lowering link mechanism, and
wherein the lifting-lowering link mechanism includes at least a first connection arm and a second connection arm as an arm which connects the bed surface-forming portion and the lower frame, the second connection arm is connected to the bed surface-forming portion side, the first connection arm is connected to the lower frame side, and the load cell is interposed between the bed surface-forming portion and the lower frame.

14. The bed having a load detection function according to claim 1,
wherein the load cell is interposed between the bed surface-forming portion and the connection-support portion.

15. The bed having a load detection function according to claim 1,
wherein the load cell is interposed between the connection support portion and the leg portion.

16. The bed having a load detection function according to claim 1,
wherein the load cell is incorporated into the leg portion.

17. The bed having a load detection function according to claim 1,
wherein the leg portion includes a caster mechanism, and the load cell is incorporated into the caster mechanism.

18. A bed-load detector which measures a change of a load applied to a bed main body by the bed-load detector being attached to the bed main body and detects a state of a user on a bed surface of the bed main body,
wherein the bed main body includes
a bed surface-forming portion which forms the bed surface,
a leg portion which comes into contact with an installation surface on which the bed main body is to be installed, and
a connection-support portion which connects the bed surface-forming portion and the leg portion such that the bed surface-forming portion is positioned above the installation surface, and transmits a load from the bed surface-forming portion to the leg portion,
wherein the load detector includes a load cell which measures strain generated when a load is applied to the bed main body,
wherein the load cell is configured to be placed at a site which is positioned at any location on a load transmission path extending from the bed surface-forming portion to the installation surface via the connection-support portion, and at which a load from the bed surface-forming portion side is received and the load is transmitted to a configuration member of the installation surface side,
wherein the load cell includes a load-receiving member which receives a load from the bed surface-forming portion side, and a base body which is separated from the load-receiving member and to which the load from the load-receiving member is applied, and
wherein the base body is configured to include an action portion with which the load-receiving member comes into contact and on which a load from the load-receiving member acts, an operating portion which is strain-deformed by the load applied to the action portion, a strain sensor which is attached to the operating portion, and an attachment portion which is continuous with the operating portion and is fixed to the configuration member of the installation surface side in the bed main body.

19. The bed-load detector according to claim 18,
wherein a bearing portion which rotatably supports a spindle, which is provided in the load transmission path of the bed main body and has a substantially horizontal axial line, is formed on the load-receiving member of the base body.
